(19) United States
(12) Patent Application Publication
(10) Pub. No.: US 2005/0123966 A1
(43) Pub. Date: Jun. 9, 2005

(54) Diagnostic and prognostic methods and compositions for seizure- and plasticity-related disorders

(76) Inventors: Thomas P. Sutula, Madison, WI (US); Sohail A. Qureshi, Karachi (PK)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/981,938

(22) Filed: Nov. 5, 2004

Related U.S. Application Data
(60) Provisional application No. 60/517,669, filed on Nov. 5, 2003.

(51) Int. Cl. C12Q 1/68; G01N 33/53; G01N 33/567; C07H 21/04; C07K 16/40

(52) U.S. Cl. 435/6; 435/7.2; 530/388.1; 536/23.2

(57) ABSTRACT

A number of molecules, including genes, gene fragments and encoded gene products which play key roles in function and outcome after initial neuronal injury are disclosed. The disclosed molecules include genes with an association with neural plasticity. The disclosed molecules, as well as the expression patterns associated with these molecules as a group, are used to advantage in a variety of assays for the identification of therapeutic agents to treat epilepsy and other conditions that also involve plasticity of neural circuits, such as stroke, trauma and neurodegenerative diseases. Compositions comprising the disclosed molecules and diagnostic methods, prognostic agents, and test kits in connection with such conditions are also disclosed.

This claims benefit of U.S. Provisional Application No. 60/517,669, filed Nov. 5, 2003, the entire contents of which are incorporated by reference herein.

Pursuant to 35 U.S.C. §202(c), it is acknowledged that the U.S. government has certain rights to the invention described herein, which was made in part with funds from the National Institutes of Health, Grant Number NS25020.

### FIELD OF THE INVENTION

This invention relates to the field neural plasticity and seizure-related disorders, such as epilepsy. More particularly, the invention provides novel diagnostic and prognostic methods and compositions based on unique profiles of gene expression that are associated with various forms of seizure-related and other disorders of the central nervous system.

### BACKGROUND OF THE INVENTION

Certain scientific publications and patents are referred to in the specification. Each of these publications is incorporated by reference herein in its entirety.

Over the past decade, considerable progress has been made in understanding how seizures that occur in the condition of temporal lobe epilepsy induce permanent structural and functional modifications in neural circuits. This research has revealed that repeated seizures induce a predictable sequence of cellular responses which include alterations in receptor-mediated excitation and inhibition, cell death, and growth of new axonal connections that reorganize neural circuits in the hippocampus. (for reviews, see Sutula T: Secondary Epileptogenesis, Kindling, And Intractable Epilepsy: A Reappraisal From The Perspective Of Neural Plasticity. In Brain Plasticity and Epilepsy, Academic Press, ed. J. Engel, Jr.; P. Schwartzkroin, S. Moshe, D. Lowenstein, International Review of Neurobiology 45:355-379, 2001; Pitkänen A, Sutula, T: Is Epilepsy A Progressive Disorder?—Prospects For New Therapeutic Approaches In Temporal Lobe Epilepsy. Lancet Neurology 1(3): 173-181, 2002; Sutula, T, Hagen, J, Pitkänen A Do Seizures Damage the Brain? Current Opinion in Neurology 16:189-195, 2003). These seizure-induced cellular alterations also occur in other conditions associated with neuronal injury, such as stroke, trauma, and neurodegenerative diseases. In contrast to the relatively well-characterized sequence of cellular alterations induced by seizures, relatively little is known about the biochemical pathways which are activated by repeated seizures, and thereby give rise to these neuronal alterations and the resulting functional consequences, including memory dysfunction and increased susceptibility to additional seizures. As only a fraction of patients who experience a seizure develop epilepsy, the biochemical changes that accompany a single seizure are themselves insufficient to cause epilepsy, and repeated seizures or sustained seizures, as in status epileptics, are necessary for the establishment of permanent changes in the hippocampal circuitry.

Both limbic kindling and kainate administration in rats serve as excellent experimental models of temporal lobe epilepsy because seizures induced by these stimuli lead to cell death and other cellular alterations in the temporal lobe similar to those observed in humans (Lynch et al., Curr. Opin. Neurol. 9: 97-102,1996; Pitkänen A, Sutula, T, 2002, Sutula T, Hagen J, Pitkänen A, 2003, supra). Seizures release glutamate, which activates postsynaptic receptors that can be distinguished by binding to glutamate analogs referred to as AMPA, kainate and NMDA. These receptors are linked to signal transduction pathways by complex transmembrane processes that ultimately influence the expression of specific genes. For example, the NMDA receptor increases the influx of Ca²⁺, activating various protein kinases/phosphatases which induce the expression of specific immediate early genes (IEGs). The IEGs may encode for proteins, which subsequently participate in the activation of effector genes whose protein products are ultimately responsible for causing long-term adaptive changes in the hippocampal circuitry. Identification and characterization of genes induced by seizures would advance the art by providing a foundation for genetic methods of diagnosing diseases that modify neuronal circuitry.

Genes induced by seizures include so-called late or immediate early genes (IEGs). Examples of IEGs include genes encoding factors like c-Fos, c-Jun, JunD, and Fos-related antigen (FRA), which are components of the AP-1 complex. AP-1 is a sequence specific transcription factor and induces the expression of other target genes, such as apo 1 (also known as Fas- or cd95-receptor) (Herdegen et al., J. Neurosci. 18: 5124-5135, 1998), gap43 (Weber and Skene, J. Neurosci. 18: 5264-5274, 1998), and NGF (Hengerer et al., Proc. Natl. Acad. Sci. USA 87: 3899-3903, 1990). Expression of these genes has been shown to promote cell death and sprouting of mossy fiber axons in the dentate gyrus, a region of the hippocampus involved in epilepsy and memory (Adams et al., J. Neurosci. 17: 5288-5296, 1997; Ashkenazi and Dixit, Science 281: 1305-1308, 1998; Benowitz and Routtenberg, Trends in Neurosci. 20: 84-91, 1997). For example, up-regulation of c-fos and c-jun has been associated with cell death (Tong et al., J. Neurochem. 71: 447-459, 1998), and mossy fiber sprouting from the granule cells has been associated with the activation of another target gene, gap 43 (Benowitz and Routtenberg, 1997, supra). Because gap 43 transcription is induced after IEG expression, and because its promoter encompasses an AP-1 recognition element (Weber and Skene, 1998, supra), it is thought that gap 43 expression is induced by AP-1.

The regulation of gap 43 expression by transcription mechanisms is an example of how activity-dependent and seizure-induced gene expression may contribute to long-term cellular alterations in neural circuits associated with epilepsy. Other effector genes whose products are also likely to influence epileptogenesis and have other long-term consequences on brain function are yet to be identified. Because cell death, axon sprouting, and rearrangements of connectivity in neural circuits (processes referred to herein collectively as “neural plasticity”) occur not only in epilepsy, but in disorders such as stroke, trauma, and neurodegenerative diseases, the genes identified in epileptogenesis influencing cellular processes that also occur in these other conditions may contribute to functional abnormalities in these conditions. Because some of these cellular alterations may also play a role in adaptation of neural circuits to injury, the underlying gene expression may also play a role in recovery of function.

For these reasons, a need exists for the identification of genes and their encoded proteins that participate in epiletogenesis and other forms of neural circuit plasticity in the central nervous system that follow neuronal injury. The molecules so identified may be used to advantage in the design of novel diagnostic agents for the identification and characterization of conditions that involve plasticity of neural circuits, including epilepsy, stroke, trauma, addiction, pain, and neurodegenerative diseases.

### SUMMARY OF THE INVENTION

In accordance with the present invention a number of genes, some of which are novel, have been identified by their increased expression as a result of seizure. It is believed that these genes and their proteins play key roles in seizure-induced neural circuit plasticity, including epileptogenesis, and other conditions accompanied by neuronal death, axon sprouting, gliosis, and plasticity-related alterations in the central nervous system that contribute to adverse function and outcome after initial neuronal injury. The genes so identified fall into two broad categories. The first category comprises known genes whose association with neural plasticity had heretofore been unappreciated. The second category comprises nucleic acid segments with no known homology to previously identified sequences. Thus, this category is believed to include one or more novel genes.

In accordance with the invention, these genes, gene fragments and encoded gene products, as well as the expression patterns associated with the group of genes as a whole, are used to advantage in a variety of assays for the identification of therapeutic agents to treat epilepsy and other conditions that also involve plasticity of neural circuits, such as stroke, trauma and neurodegenerative diseases. They are also employed as novel diagnostic and prognostic agents in connection with such conditions.

In one aspect of the invention, compositions of matter comprising a collection of two or more probes for detecting expression of two or more seizure-induced genes are provided. In certain presently preferred embodiments, the probes comprise one or more of: a) oligonucleotides or polynucleotides that specifically hybridize to two or more of SEQ ID NOS: 1-48; or b) polypeptide binding agents that specifically bind to polypeptides produced by expression of two or more nucleic acid molecules comprising sequences selected from the group consisting of SEQ ID NOS: 1-48. In preferred embodiments, the composition comprises a collection of five or more, or ten or more probes for detecting expression of five or more, or ten or more seizure-induced genes, respectively. In certain embodiments, the compositions of matter comprise probes which are affixed to a solid support at one or more known locations. In some presently preferred embodiments, the polypeptide binding agents are antibodies.

The invention also provides, in another of its several aspects, compositions and test kits to facilitate practice of the many screening, diagnostic and prognostic assays of the invention. Presently preferred are test kits for analyzing expression of seizure-induced genes. The test kits preferably comprise a container containing a collection of two or more probes for detecting expression of two or more seizure-induced genes. The probes can comprise: a) oligonucleotides or polynucleotides that specifically hybridize to two or more of SEQ ID NOS: 1-48; or b) polypeptide binding agents that specifically bind to polypeptides produced by expression of two or more nucleic acid molecules comprising sequences selected from the group consisting of SEQ ID NOS: 1-48. The kits preferably further comprise instructions for performing a gene expression assay. In some presently preferred embodiments, the test kits comprise a collection of five or more probes for detecting expression of five or more seizure-induced genes, or for example, a collection of ten or more probes for detecting expression of ten or more seizure-induced genes. In other embodiments, the test kits comprise probes affixed at known locations to a solid support. The use of polypeptide binding agents which are antibodies is contemplated herein for use with the kits.

In another of its several aspects, the invention encompasses devices for detecting expression of a plurality of seizure-induced genes, the devices comprising a solid support to which is affixed an array comprising a plurality of probes specific for the products of transcription of the seizure induced genes, or translation products thereof, wherein the probes comprise either: a) a plurality of oligonucleotides or polynucleotides, each of which specifically hybridizes to a different sequence selected from the group consisting of SEQ ID NOS: 1-48, or b) a plurality of polypeptide binding agents, each of which specifically binds to a different polypeptide or fragment thereof produced by expression of a nucleic acid molecule comprising a sequence selected from the group consisting of SEQ ID NOS: 1-48. In one embodiment, the device comprises polypeptide binding agent probes which are antibodies.

In another aspect of the invention, isolated nucleic acid molecules are provided, the molecules comprising a seizure-induced gene, mRNA, or cDNA produced from the seizure induced gene. The nucleic acid molecules comprise a sequence selected from the group consisting of SEQ ID NOS: 29-48.

Also provided in an aspect of the invention are polypeptides encoded by the nucleic acid molecules described above.

In another of its several aspects, the invention provides methods for measuring the effect of a test compound on activity of a polypeptide produced by expression of a seizure induced gene. Preferably, the polypeptide is selected from the group consisting of: Bach1, Interferon Related Developmental Regulator (IFRD1/PC4), Cis-Golgi SNARE p28, TFIID subunit TAFII55, FGF-Inducible gene (FIN14) product, Esterase-D or S-formylglutathione hydrolase, -L-fucosidase, Integrin 6 subunit, Macrophilin (microphilin and actin filament crosslinker protein or ACF7), Synaptogyrin 3, Metallothionine I and II, and GM3 synthase (or Sialytransferase 9). The method preferably comprises measuring a biological activity of the polypeptide in the presence or absence of the test compound, wherein a change in the biological activity in the presence of the test compound is indicative of an effect of the test compound on activity of the polypeptide.

In another aspect methods are provided for measuring the effect of a test compound on expression of a seizure-induced gene. Preferably the gene is selected from the group consisting of Bach1, MafK, Interferon Related Developmental Regulator (IFRD1/PC4), Cis-Golgi SNARE p28, TFIID subunit TAFII55, FGF-Inducible gene (FIN14), Esterase-D or S-formylglutathione hydrolase, -L-fucosidase, Integrin. 6 subunit, Macrophilin (microphilin and actin filament crosslinker protein) or ACF7, Synaptogyrin 3, Metallothionine I and II, and GM3 synthase or Sialytransferase 9. The method preferably comprises measuring production of transcription or translation products produced by expression of the gene in the presence or absence of the test compound, wherein a change in the production of transcription or translation products in the presence of the test compound is indicative of an effect of the test compound on expression of the gene. Methods are also provided wherein the gene expression is measured by providing a DNA construct comprising a reporter gene coding sequence operably linked to transcription regulatory sequences of the seizure-induced gene, and measuring formation of a reporter gene product in the presence or absence of the test compound. In certain presently preferred embodiments, the gene is located within a cultured cell and the cultured cell is contacted with the test compound. In certain embodiments the cultured cell is a cell line derived from primary neuronal or glial cells. Methods are also provided wherein the gene is located within a mammalian subject and the test compound is administered to the subject.

Another aspect of the invention provides methods for measuring the effect of a test compound on the expression profile of a plurality of seizure-induced genes comprising two or more of SEQ ID NOS: 1-48. The method preferably comprises measuring production of transcription or translation products produced by expression of the plurality of genes in the presence and/or absence of the test compound, wherein a change in the production of transcription or translation products of any of the genes in the presence of the test compound is indicative of an effect of the test compound on the expression profile of the plurality of seizure-induced genes. In certain embodiments, the plurality of genes is located within a cultured cell and the cultured cell is contacted with the test compound. Methods also are provided in which the cultured cell is a cell line derived from primary neuronal or glial cells. In some embodiments the cells are treated with kainic acid prior to, concurrently with, or after exposure to the test compound, while in other embodiments the cells are further treated with an NMDA receptor antagonist prior to, concurrently with, or after exposure to the test compound. Also provided are methods wherein the gene is located within a mammalian subject and the test compound is administered to the subject. In certain presently preferred embodiments the mammalian subject is an animal and the animal is subjected to inducement of a seizure prior to, concurrently with, or after administration of the test compound. Also useful are methods wherein the animal is further treated with an NMDA receptor antagonist prior to, concurrently with, or after exposure to the test compound. In some embodiments the mammalian subject is an animal and the animal is bred or genetically modified to exhibit slow kindling as a result of repeated seizure inducement. In other embodiments of the methods, the mammalian subject is an animal and the animal is bred or genetically modified to exhibit fast kindling as a result of repeated seizure inducement. Also provided are methods as described which are adapted to establish an NMDA receptor-dependent and an NMDA receptor-independent gene expression profile in the absence of the test compound, wherein the animal is subjected to inducement of a seizure with or without administration of an NMDA receptor antagonist, and production of transcription or translation products produced by expression of each of a plurality of seizure-induced genes having sequences comprising SEQ ID NOS: 1-48 is measured, and the differences in production of transcription or translation products of each of the genes in the presence versus absence of the NMDA inhibitor is observed, thereby generating the NMDA receptor-dependent and NMDA receptor-independent gene expression profile of the seizure-induced genes.

In another of its aspects, the invention provides methods to diagnose or develop a prognosis for a subject who has had a seizure. In an exemplary embodiment, the method comprises the steps of:
a) obtaining a sample of neural cells from the subject;
b) measuring production of transcription or translation products produced by expression of two or more of a plurality of seizure-induced genes having sequences comprising SEQ ID NOS: 1-48;
c) determining whether any of the transcription or translation products of the seizure-induced genes is elevated as compared with a known control, wherein an elevation in transcription or translation products of any of the seizure-induced genes is indicative of the subject having had a seizure.

In some embodiments, the methods further comprise assessing whether the increased seizure-induced gene expression in the subject is characterized as NMDA-dependent or NMDA-independent gene expression.

Also provided among the various aspects of the invention are methods of determining if a test compound affects NMDA-dependent, but not NMDA-independent seizure-induced gene expression in an animal subject. Presently preferred are methods comprising the steps of:
a) establishing an NMDA receptor-dependent and an NMDA receptor-independent gene expression profile in the absence of the test compound by:
   i) inducing a seizure in one animal in the presence of an NMDA receptor antagonist and inducing a seizure in another animal in the absence of an NMDA receptor antagonist;
   ii) obtaining samples of transcription or translation products from neural cells of each animal; and
   iii) identifying transcription or translation products that are differentially produced in the neural cells of animals induced to seizure in the presence of the NMDA receptor antagonist, as compared to in the absence of the NMDA receptor antagonist, thereby establishing the NMDA receptor-dependent and NMDA-receptor independent gene expression profiles and identifying a population of NMDA receptor-associated seizure-induced genes;
b) repeating step a) in the presence of the test compound, wherein differences in expression of one or more of the NMDA receptor-associated seizure-induced genes in the presence of the test compound is indicative that the test compound affects NMDA receptor-dependent but not NMDA receptor-independent seizure-induced gene expression.

In certain embodiments, the gene expression profiles are generated by interrogating the samples of transcription or translation products on an array that represents all expressed genes in the animal. In other embodiments, the gene expression profiles are generated by interrogating the samples of transcription or translation products on an array that represents nucleic acids comprising SEQ ID NOS: 1-48 or polypeptides encoded by genes comprising SEQ ID NOS: 1-48. In presently preferred methods, the animal is a rat. Also in presently preferred methods the NMDA receptor antagonist is MK801.

Other features and advantages of the present invention will be further understood by reference to the detailed description and the examples that follow.

### DETAILED DESCRIPTION OF ILLUSTRATIVE EMBODIMENTS

I. Definitions:

The following definitions are provided to facilitate an understanding of the present invention:

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program.

A fragment” or “portion” of an epilepsy-related or neural plasticity related polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. Fragments of the epilepsy and neural plasticity-related polypeptide sequence, antigenic determinants, or epitopes are useful for eliciting immune responses to a portion of the epilepsy and neural plasticity-related protein amino acid sequence.

Different “variants” of the epilepsy and neural plasticity-related polypeptides exist in nature. These variants may be alleles characterized by differences in the nucleotide sequences of the gene coding for the protein, or may involve different RNA processing or post translational modifications. The skilled person can produce variants having single or multiple amino acid substitutions, deletions, additions or replacements. These variants may include inter alia: (a) variants in which one or more amino acids residues are substituted with conservative or non conservative amino acids, (b) variants in which one or more amino acids are added to the polypeptide, (c) variants in which one or more amino acids include a substituent group, and (d) variants in which the polypeptide is fused with another peptide or polypeptide such as a fusion partner, a protein tag or other chemical moiety, that may confer useful properties to the epilepsy and neural plasticity-related polypeptide, such as, for example, an epitope for an antibody, a polyhistidine sequence, a biotin moiety and the like. Other epilepsy and neural plasticity-related polypeptides of the invention include variants in which amino acid residues from one species are substituted for the corresponding residue in another species, either at the conserved or non conserved positions. In another embodiment, amino acid residues at non conserved positions are substituted with conservative or non conservative residues. The techniques for obtaining these variants, including genetic (suppressions, deletions, mutations, etc.), chemical, and enzymatic techniques are known to the person having ordinary skill in the art. To the extent such allelic variations, analogues, fragments, derivatives, mutants, and modifications, including alternative nucleic acid processing forms and alternative post translational modification forms result in derivatives of the epilepsy and neural plasticity-related polypeptide that retain any of the biological properties of the epilepsy and neural plasticity-related polypeptide, they are included within the scope of this invention.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage, artificial chromosome (BAC, YAC) or virus, that is capable of replication largely under its own control. A replicon may be either RNA or DNA and may be single or double stranded.

A “vector” is a replicon, such as a plasmid, cosmid, bacmid, phage, artificial chromosome (BAC, YAC) or virus, into which another genetic sequence or element (either DNA or RNA) may be inserted so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to sequences, primers and probes of the present invention, and is defined as a nucleic acid molecule comprised of two or more ribo or deoxyribonucleotides, preferably more than three. The exact size of the oligonucleotide will depend on various factors and on the particular application and use of the oligonucleotide.

The term “probe” as used herein refers to either a probe for a nucleic acid or a probe for a protein. When used in connection with nucleic acids, a “probe” refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single stranded or double stranded. The exact length of the probe will depend upon many factors, including temperature, source of probe and method of use. For example, for diagnostic applications, depending on the complexity of the target sequence, the oligonucleotide probe typically contains 10-100, preferably 15-50, more preferably 15-25 nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non complementary nucleotide fragment may be attached to the 5′ or 3′ end of the probe, with the remainder of the probe sequence being complementary to the target strand. Alternatively, non complementary bases or longer sequences can be interspersed into the probe, provided that the probe sequence has sufficient complementarity with the sequence of the target nucleic acid to anneal therewith specifically. When used in connection with a protein, a “probe” is a protein binding substances capable of specifically binding a particular protein or protein fragment to the substantial exclusion of other proteins or protein fragments. Such binding substances may be any molecule to which the protein or peptide specifically binds, including DNA (for DNA binding proteins), antibodies (as described in greater detail herein), cell membrane receptors, peptides, cofactors, lectins, sugars, polysaccharides, cells, cell membranes, organelles and organellar membranes.

The term “specifically hybridize” refers to the association between two single stranded nucleic acid molecules of sufficiently complementary sequence to permit such hybridization under pre determined conditions generally used in the art (sometimes termed “substantially complementary”). In particular, the term refers to hybridization of an oligonucleotide with a substantially complementary sequence contained within a single stranded DNA or RNA molecule of the invention, to the substantial exclusion of hybridization of the oligonucleotide with single stranded nucleic acids of non complementary sequence.

The term “primer” as used herein refers to an oligonucleotide, either RNA or DNA, either single stranded or double stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield an primer extension product. The primer may vary in length depending on the particular conditions and requirement of the application. For example, in diagnostic applications, the oligonucleotide primer is typically 15 25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non complementary nucleotide sequence may be attached to the 5′ end of an otherwise complementary primer. Alternatively, non complementary bases may be interspersed within the oligonucleotide primer sequence, provided that the primer sequence has sufficient complementarity with the sequence of the desired template strand to functionally provide a template primer complex for the synthesis of the extension product.

Amino acid residues described herein are preferred to be in the “L” isomeric form. However, residues in the “D” isomeric form may be substituted for any L amino acid residue, provided the desired properties of the polypeptide are retained. All amino acid residue sequences represented herein conform to the conventional left-to-right amino terminus to carboxy terminus orientation.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “substantially pure” refers to a preparation comprising at least 50 60% by weight of a given material (e.g., nucleic acid, oligonucleotide, protein, etc.). More preferably, the preparation comprises at least 75% by weight, and most preferably 90 95% by weight of the given compound. Purity is measured by methods appropriate for the given compound (e.g. chromatographic methods, agarose or polyacrylamide gel electrophoresis, HPLC analysis, and the like).

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino or carboxy terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c myc epitope, transmembrane epitope of the influenza A virus hemaglutinin protein, protein A, cellulose binding domain, calmodulin binding protein, maltose binding protein, chitin binding domain, glutathione S transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

As used herein, the terms “reporter,” “reporter system”, “reporter gene,” or “reporter gene product” refer to an operative genetic system in which a nucleic acid comprises a gene that encodes a product that when expressed produces a reporter signal that is a readily measurable, e.g., by biological assay, immunoassay, radioimmunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, refer to any method or means by which a nucleic acid is introduced into a cell or host organism and may be used interchangeably to convey the same meaning. Such methods include, but are not limited to, transfection, electroporation, microinjection, PEG-fusion and the like.

A “clone” or “clonal cell population” is a population of cells derived from a single cell or common ancestor by mitosis.

A “cell line” is a clone of a primary cell or cell population that is capable of stable growth in vitro for many generations.

An “immune response” signifies any reaction produced by an antigen, such as a viral antigen, in a host having a functioning immune system. Immune responses may be either humoral in nature, that is, involve production of immunoglobulins or antibodies, or cellular in nature, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured both in in vitro and in various cellular or animal systems. Such immune responses may be important in protecting the host from disease and may be used prophylactically and therapeutically.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunloglobulin molecule such as those portions known in the art as Fab, Fab′, F(ab′)2 and F(v).

As used herein, the term “living host” means any non human autonomous being.

As used herein, the term “subject” or “patient” refers to both humans and animals, unless specified that the “subject” or “patient” is an animal or a human.

The term “epilepsy” describes a chronic disorder characterized by paroxysomal brain dysfunction due to excessive neuronal discharge and is usually associated with some alteration of consciousness.

The phrase “neural plasticity” as used herein refers to controlled biochemical processes related to activity-dependent alterations in receptors, axon sprouting, programmed cell death and rearrangements in connectivity of neural circuitry.

II. Description:

One remarkable aspect of the brain is its ability to reorganize itself in response to experience. The brain can, in fact, change the structural and functional characteristics of its cells (neurons) and even “rewire” the connections between those cells as needed. This general phenomenon is known as neural plasticity and is broadly involved in brain function, affecting such fundamental processes as learning, memory and disease states. Neural plasticity is also involved in several disorders or malfunctions of the human central nervous system, including epilepsy and the consequences of stroke, brain trauma and neurodegenerative diseases. For example, temporal lobe epilepsy (TLE) is associated with characteristic cellular and functional alterations in neural circuitry of the temporal lobe, which include hippocampal sclerosis, mossy fiber sprouting and memory disorders.

In the kindling model of epilepsy and neural plasticity, initially sub-convulsive stimuli result in gradual intensification of epileptiform seizures that subsequently become spontaneous. Once kindled seizures have been repeatedly induced, increased susceptibility to repeated seizures, and eventually spontaneous seizures, is permanent. Kindling can be induced either by electrical or chemical activation of a variety of neuronal pathways in diverse species such as amphibians, mammals and primates. Intraperitoneal or subcutaneous administration kainic acid, an analog of glutamate, also causes similar morphological and anatomical changes in the hippocampal neurons that are observed in the kindling model and also results in the animal becoming epileptic.

Previously, certain genes encoding for transcription factors such as c-Fos, c-Jun, and Zif/268 have been shown to be induced rapidly in response to seizure evoking stimuli in chronic models of TLE such as kainic acid or limbic kindling. Using differential display PCR, RT-PCR and in situ hybridization analysis, the inventors have identified a significant number of additional genes, whose expression is rapidly induced in hippocampal neurons after the administration of kainic acid.

A. Polynucleotides Representing Genes and Gene Fragments Induced in Neurons Following Administration of Kainic Acid:

Nearly 50 genes have been identified in accordance with the present invention, whose functions are closely associated with the phenomenon of neural plasticity. The association is determined by comparing expression of the genes in normal brains and in brains that have been induced by kainic acid to undergo specific modifications that ultimately lead to the epileptic state. The genes so identified fall into two broad categories. The first category comprises known genes whose association with neural plasticity had heretofore been unappreciated. These include the following: Bach1, MafK, Interferon Related Developmental Regulator (IFRD1/PC4), Cis-Golgi SNARE p28, TFIID subunit TAFII55, FGF-Inducible gene (FIN14), Esterase-D or S-formylglutathione hydrolase, -L-fucosidase, Integrin 6 subunit, Macrophilin (microphilin and actin filament crosslinker protein) or ACF7, Synaptogyrin 3, Metallothionine I and II, and GM3 synthase or Sialytransferase 9. Each of the proteins encoded by these genes is described in greater detail below.

The second category comprises nucleic acid segments with no known homology to previously identified sequences. Thus, this category is believed to include one or more novel genes.

In total, segments of 48 kainic acid-induced genes from rat hippocampii were obtained by employing the differential display protocol of Liang and Pardee (Mol. Biotechnol. 10: 261-267, 1998). The DNA sequences of the 19 gene segments previously identified (SEQ ID NOS: 1-19) as well as the GenBank accession numbers of their respective homologs are listed below. Also listed are nine sequences (SEQ ID NOS: 20-28) that correspond to previously identified EST sequences. Twenty sequences (SEQ ID NOS: 29-48) with no known homology to previously identified sequences also are set forth below.

| | |
|---|---|
| gccatggtaatcctgctcagtacgagaggaacngcag | SEQ ID NO: 1 |
| tctagactattggtgtatgtgcttggctgaggagcca | |
| atggggccacagtaccatctgtggat | |

similar to gb|AF132045| Rat foocen-m2 mRNA

gb|JO4633| mouse heat shock protein 86 mRNA

gi|22129752|ref|NM_{—}147146.1|Rattus norvegicus small androgen receptor-interacting protein (sarip),

| | |
|---|---|
| actgttttactctgcagcccaggaaagcccacatcgc | SEQ ID NO: 2 |
| ctgactgctcgtatgtgtgtaggaccgacaggaatag | |
| agcccgtgagtccagcagaatcctg | |

homologous to dbj|D86603|D86603 Mouse mRNA for Bach protein 1.

| | |
|---|---|
| agcttggtgaacttagataatttttgatgtagggatg | SEQ ID NO: 3 |
| ggttatattttaatttaatgtacagtgttacaaatta | |
| atgagttctttattcctgtaaaaata | |

Homologous to: gb|J04511|RATIRPRA Rattus norvegicus IFRD1 (PC4) mRNA, interferon related, developmental regulator, nerve growth factor-inducible, complete; emb|X17400|MMTIS7M mRNA for TIS7 protein;

emb|V00756|MMIFR2 mRNA fragment for mouse interferon (type 2).

Ifrd1—Interferon-related developmental regulator 1

| | |
|---|---|
| cgtaagtaggcagaatcaacccatgcttttattttca | SEQ ID NO: 4 |
| tggcatataaaaattttatatgaaggtattgatgtac | |
| agtgctatccccatggctgtaagagagg | |

Similar to gi|22901891|gb|AF536980.1| Homo sapiens cAMP-specific phosphodiesterase (PDE4D) mRNA, untranslated region, partial sequence

gb|AI029492|AI029492 UI-R-C0-iw-f-01-0-UI.s1 UI-R-C0 Rattus norvegicus cDNA clone UI-R-C0-iw-f-01-0-UI 3′.

| | |
|---|---|
| aagcttccagcaacaaaccaataacatactactcaac | SEQ ID NO: 5 |
| ataatactcatttcactagccataaaatccggactag | |
| ccccattccactactgactacccgaa | |

Similar to gb|U49099|RNU49099 Rattus norvegicus cis-Golgi p28 (p28) mRNA;

Reported in: Science 1996 May 24;272(5265):1161-3.

| | |
|---|---|
| agcagcagcagcgaagatgaagaagatgtaaacatga | SEQ ID NO: 6 |
| ttgacactgaagaggatctggagagacagctacagga | |
| caagctaaatgagtc | |

Homologous to

gi|6755711|ref|NM_{—}011901.1| Mus musculus TAF7 RNA polymerase II, TATA box binding protein

(TBP)-associated factor, 55 kDa (Taf7), mRNA

gi|16807132|gb|BC013397.1| Homo sapiens, similar to TAF7 RNA polymerase II, TATA box binding protein (TBP)-associated factor, 55 kD, clone

| | |
|---|---|
| ttggtgaacgtaaagattgaccctcaagtcactgtag | SEQ ID NO: 7 |
| ctttagtacttgcttattgtattagtttagatgctag | |
| caccgcatgtgctctgcatatttctggttt | |

Homologous to gb|U42386|MMU42386 Mus musculus fibroblast growth factor inducible gene 14 (FIN14) mRNA.

gb|AF000982|HSAF000982 Homo sapiens dead box, X isoform (DBX) mRNA alternative transcript 2.

| | |
|---|---|
| cgacctgtctcgtgaagtcctactcgggtccacaga | SEQ ID NO: 8 |
| ttgacatactaattgatcaggggaaagatgacggag | |
| ttctctcgaatggccagttactcctgacaacttcag | |
| catcgctgcctgcacagaaaagaaaatccctgttgt | |
| ttttt | |

Homologous to dbj|ABO25408.1| mouse mRNA for sid478P gb|M13450|HUMETRD Human esterase D mRNA, 3′end.

gi|20961915|ref|XM_{—}125266.1| Mus musculus similar to Esterase D (LOC215667), mRNA

| | |
|---|---|
| ggaaaggaagctactttattcacgataatccttaaga | SEQ ID NO: 9 |
| gacagcagcacactgggccctctatccttgagctcca | |
| gcgatcagttcacttttgtcagctttagagtccaggc | |
| agttctccttccattcctag | |

Homologous to emb|X16145|RNALFUC Rat mRNA for liver a-L-Fucosidase.

Reported in Biochem J 1989 Dec. 15;264(3):695-701; Isolation and sequence analysis of a cDNA encoding rat liver alpha-L-fucosidase.

gb|AI409326|AI409326 EST237618 Normalized rat kidney, Bento Soares Rattus sp. cDNA clone RKIDM76 3′ end.

| | |
|---|---|
| acgatgctaggaatggaaggagaactgcctggactct | SEQ ID NO: 10 |
| aaagctgacaaaagtgaactgatcgcaggagctcaag | |
| gatagagggcccagtgtgctgctgtctcttaaggatt | |
| atcgtgaataaagtagcttcctttccaaaaaaa | |

Homologous to emb|X16145|RNALFUC Rat mRNA for liver a-L-Fucosidase.

Reported in Biochem J 1989 Dec. 15;264(3):695-701—Isolation and sequence analysis of a cDNA encoding rat liver alpha-L-fucosidase

| | |
|---|---|
| ggccttacgaatgttaacagtttcaactatacatgag | SEQ ID NO: 11 |
| aacgacacaacaacatttagtttgcttatgaacttca | |
| taaaagaacatgtttaatcttcattcttgaaatccat | |
| ttcattgcattgtcaaatgataatttcagtgtg | |

Similar to gi|20985246|ref|XM_{—}136034.1| Mus musculus similar to kelch-like 4, isoform 1 [Homo sapiens](LOC237010), mRNA

| | |
|---|---|
| cggggttatcagtgtgtttctcgtcgttgatctctcg | SEQ ID NO: 12 |
| ctcttctttccgaatccgcaccgcatggtatcgggga | |
| acactgtcatcgacctagag | |

Homologous to gi|13992590|emb|AJ312934.1|RNO312934 Rattus norvegicus partial mRNA for integrin alpha 6 subchain (inta6gene), 3′ end

emb|X69902|MMINTEG M. musculus mRNA for integrin alpha6 subunit.

Reported in Cell Adhes Commun 1993 May;1(1):33-53. Variants of the alpha 6 beta 1 laminin receptor in early murine development: distribution, molecular cloning and chromosomal localization of the mouse integrin alpha 6 subunit.

EST: gb|W34024|W34024 mb01d12.r1 Soares mouse p3NMF19.5 Mus musculus cDNA clone 318935 5′ similar to gb:X53586_rnal INTEGRIN ALPHA-6 PRECURSOR (HUMAN); gb:X69902 M. musculus mRNA for integrin alpha6 subunit (MOUSE).

| | |
|---|---|
| gaattcttattttttttaatataatttatcttgctga | SEQ ID NO: 13 |
| tatagcaagtcaagtttataaagaagatgcatttagg | |
| aataatcctaaaa | |

Homologous to dbj|AB007934|AB007934 Homo sapiens mRNA for KIAA0465 protein;

gb|AF150755| mouse microtubule-actin crosslinking factor macF mRNA;

gb|AF141968| human trebeculun-mRNA;

dbj|AB029290.1| human mRNA for actin binding protein ABP620.

Reported in DNA Res 1997 Oct. 31;4(5):345-9. Characterization of cDNA clones in size-fractionated cDNA libraries from human brain.

gb|AI072264|AI072264 UI-R-C2-ne-b-06-0-UI.s1 UI-R-C2 Rattus norvegicus cDNA clone UI-R-C2-ne-b-06-0-UI 3′.

| | |
|---|---|
| caatacatctgcatcttgaccttttgttttcctcaaa | SEQ ID NO: 14 |
| tatttcatctctgggccctgtctgttacagggttacc | |
| aggagtcaaattttatctaccataaatattcacatga | |
| aaatagtaactt | |

Homologous to dbj|AB007934|AB007934 Homo sapiens mRNA for KIAA0465 protein.

gb|AI237536|AI237536 EST234098 Normalized rat placenta, Bento Soares Rattus sp. cDNA clone RPLDA31 3′ end.

gb|AF150755| mouse microtubule-actin crosslinking factor macF mRNA;

gb|AF141968| human trebeculun-mRNA;

dbj|AB029290.1| human mRNA for actin binding protein ABP620.

| | |
|---|---|
| caatgcatttacagtctttattgtaaagacgttgcag | SEQ ID NO: 15 |
| ttgtgacaggggacactgaaaatacacaatcacggat | |
| tgttctttcagaagagagaggaatagatcccatatat | |
| acaaaa | |

Homologous to gi|26349022|dbj|AK08 1164.1| Mus musculus 10 days neonate cerebellum cDNA, RIKEN full-length enriched library, clone: B930094O21 product: synaptogyrin 3 full insert sequence

| | |
|---|---|
| gataagcttcctgcaatggtacacccagacccagcac | SEQ ID NO: 16 |
| aagatgagaagacccagcttgttattaaaagacatcc | |
| tcaagtgcatgttggttgtgtttggagtgt | |

Homologous to dbj|AB018049|AB018049 Rattus norvegicus mRNA for GM3 synthase.

| | |
|---|---|
| cctgcaaatgcaaacaatgcaaatgcacctcctgcaa | SEQ ID NO: 17 |
| gaaaagctgctgttcctgctgccccgtggtgtgcgaa | |
| gtgctcccagggctgcatctgcaaagaggcttcggac | |
| aagtg | |

Homologous gb|M11265|CRUMETIIA Chinese hamster metallothionein II gene.

gb|M11794|RATMT12C Rat metallothionein-2 and metallothionein-1 genes.

| | |
|---|---|
| cctgcaaatgcaaacaatgcaaatgcacctcctgcaa | SEQ ID NO: 18 |
| gaaaagctgctgttcctgctgccccgtggtg | |

Homologous to gi|12832641|dbj|AK002567.1| Mus musculus adult male kidney cDNA, RIKEN full-length enriched library, clone: 0610011P06 product: METALLOTHIONEIN-II (MT-II), full insert sequence

emb|V01533|MOTHIO Monkey complementary DNA coding for metallothionein.

| | |
|---|---|
| ggccttacgaatgttaacagtttcaactatacatgag | SEQ ID NO: 19 |
| aacgacacaacaacatttagtttgcttatgaacttca | |
| taaaagaacatgtttaatcttcattcttgaaatccat | |
| ttcattgcattgtcaaatgataatttcagtgtagctt | |
| cacaacaacaacacatgctccagctctt | |

Homologous to gi|20985246|ref|XM_{—}136034.1| Mus musculus similar to kelch-like 4, isoform 1 [Homo sapiens](LOC237010), mRNA

| | |
|---|---|
| ttagcagcagcagcagaataccacagcaggaaaatga | SEQ ID NO: 20 |
| agagctggcaacagcaggctctaggacatgtatataa | |
| tgcactgctattgtacagtacagagagaaagact | |

homologous to gi|12851594|dbj|AK013990.1| Mus musculus 13 days embryo head cDNA, RIKEN full-length enriched library, clone: 3110004H13 product: weakly similar to CG1057 PROTEIN (LD35644P) [Drosophila melanogaster], full insert sequence

| | |
|---|---|
| ttagcagcacgaggagaaaacgggaggatgtgtctga | SEQ ID NO: 21 |
| aaaaagcngtcctgcacagaagtattgtatttttatg | |
| aattttt | |

gb|AI102015|AI102015 EST211304 Normalized rat brain, Bento Soares Rattus sp. cDNA clone RBRBX95 3′ end.

| | |
|---|---|
| cgtacgtatacacactcacacatacaaatttattcg | SEQ ID NO: 22 |
| aattcagcaaatagacccatcttagctaaaagaaga | |
| attc | |

homologous to gi|23892935|emb|AL672290.16| Mouse DNA sequence from clone RP23-471M13 on chromosome X, complete sequence

| | |
|---|---|
| ccatgcatttgctaatttcattaccattaaagtttgt | SEQ ID NO: 23 |
| tgcaacacaagtttccagaaattctaattttgtcttt | |
| taaacatttaccatcatcagtgaacacttatcagtga | |
| aaggttcagaatgttcatttttcaggaacacagctaa | |
| atatccaagcc | |

homologous to gi|26099111|dbj|AK080259.1| Mus musculus adult male aorta and vein cDNA, RIKEN full-length enriched library, clone: A530094104 product: WD40 protein Ciaol, full insert sequence

| | |
|---|---|
| tccatgggaaacgaagcggaccagagtgcctatcacc | SEQ ID NO: 24 |
| acatctgggctatcagtagactgtccacaccttgtct | |
| tgctcttagcccctcggactccatccgtgttgccccc | |
| tggtgtccacggaagactgtacagaaccattgtttat | |
| ttatagccactctcccattggatttttaagaaaacag | |
| gataaacgatgatgttt | |

homologous to gi|20502869|gb|AF469109.1| Mus musculus SDS3 mRNA, complete cds

| | |
|---|---|
| acgttatataacaaatcaagtccatagtgagaacatt | SEQ ID NO: 25 |
| attatggagaaatatgcaatggcttctgcataggaca | |
| ttcatatctatttgaagaagcttgtgatcc | |

Homologous to gi|25168711|emb|AL928598.61 Mouse DNA sequence from clone RP23-326120 on chromosome 2, complete sequence

| | |
|---|---|
| aaaaacaacaggagcaacaatgaaaaactaaaagtaa | SEQ ID NO: 26 |
| agacggtgacggatcagggcactaaaaaacttacagt | |
| tgttgtataagctgcttctgggtcgt | |

homologous to gi|21954880|gb|AC117439.5| Homo sapiens 3 BAC RP11-183P11 (Roswell Park Cancer Institute Human BAC Library) complete sequence

| | |
|---|---|
| aagcttggtgaactggtagaagtgcgtcctgggagtg | SEQ ID NO: 27 |
| aaatgaggtgtcactggcaggtgagtgaaaacagcct | |
| gagtcttcccttggctagtaagccc | |

homologous to gi|26090014|dbj|AK043844.11 Mus musculus 10 days neonate cortex cDNA, RIKEN full-length enriched library, clone: A830039H05 product: unknown EST, full

| | |
|---|---|
| atcattaaaacgccccagactttatttagcattagtt | SEQ ID NO: 28 |
| agcaaagctggcttaacaaagtgaaagctgaaagttc | |
| acctagttaggaaaaggaacttacagatgagaaatga | |
| aacactaggaaactcagaagcgacaaatccaagtggt | |
| aggagaaagcaaatgccctatacgtac | |

homologous to gi|23462923|gb|AC111014.6| Mus musculus chromosome 10 clone RP24-371P8, complete sequence

The following sequences are novel and do not have significant homology to previously identified sequences.

| | |
|---|---|
| ccctaatgtattgtgtagcaatacatggaaaggatca | SEQ ID NO: 29 |
| tggaagcatgacacaagatgtaatttaaattttcaaa | |
| agtcttatggttgtcatgaagagaga | |
| tacgtattgagttatgatttcaaatgctaccttggga | SEQ ID NO: 30 |
| agttccaatggagatttgagttgaacagaaaaataca | |
| gtttcaggaacgtttcgttcttcttag | |
| Ggtgaactgtaagtgggaaaaacagtgtaaaaatcag | SEQ ID NO: 31 |
| agatgatgtttaaaaaannttaaggctcttttgggac | |
| atgtgtgatatgctaggaaattaatttt | |
| ggtgaacagtaaaacaaatcaatagaacagaatggag | SEQ ID NO: 32 |
| taatgaaccctcataaatacagtccacaaatcattta | |
| caaagagcaaaggtgatctttccagc | |
| cttgcccacgtctcatacaaatgttctttgtcacttt | SEQ ID NO: 33 |
| gtgagcagttgtgtgtatcctagtcttactcctacag | |
| cgtcaacatttgggaagccgagtcag | |
| aagcttgctgctagcaaaacaccagtagacacaaaat | SEQ ID NO: 34 |
| aaaaataaagtataaaaaagaagacagggggctagag | |
| atatggctcagcgattgagagcactg | |
| atgcaaaggaaaagacgtgagggaaacggttgagcat | SEQ ID NO: 35 |
| aatcaaaaaacacaatcctgnnccttaggttcctgta | |
| ctgtacagcctcacatccagcccataggattcagtga | |
| ggagttaggtcttga | |
| ttggtgaacccagacggagataactcagaatgcttgt | SEQ ID NO: 36 |
| aaagtcagtccccacctgccccaaattttt | |
| ctgtcacagttatgatcaattccccgggaaagaacag | SEQ ID NO: 37 |
| cgcttttctaatttacaacagtcctccaaccgtcttt | |
| acttaatcacgttttgaaatttccgtgtaaccgttta | |
| ttaagcttct | |
| tttttttagttccaagtgtctggttatttcgcaatat | SEQ ID NO: 38 |
| cttttcggcgccgggcttgattagttgtgatatgnga | |
| gtgaccgataaa | |
| tcgtcgtcgtcgtcaaggacatccaggtacccgagaa | SEQ ID NO: 39 |
| accacttgtcgtgtgtggtaacaccgtcggcggaagt | |
| ctcacgggttcggttcacaaaccaccctctttgttct | |
| cagc | |
| gtatctgcccaactaagtactttaacaaaaatccatc | SEQ ID NO: 40 |
| gagcaaaccaaagnncnaagaagtcgaatttaggaaa | |
| agcaa | |
| ccctttacaacgagatccatttaatctttaaaaaaaa | SEQ ID NO: 41 |
| agattatgatactttttccgttctcaaaaagagccag | |
| tcgtatgtaaaatataaatgaaacaggaaaatgacgt | |
| ggggtctcttggaaaatactaattcccactaaaccg | |
| aacctgtgtcttaggcacactacgacatgggtagggc | SEQ ID NO: 42 |
| ctaaatttgattacctctcatttatttattttcatct | |
| aaacnngagaacttttt | |
| caattttgcggggtctgaaataaatcgtaatcaatcg | SEQ ID NO: 43 |
| tttcgaccgaattgtttcactttcgactttcaagtga | |
| tcaatccttttccttgaatgtctactctttac | |
| cttattttagtacaagtaaataaaaaaangacgtaga | SEQ ID NO: 44 |
| atcttccgtattttaatttatacaacgttacattatt | |
| taagtaggtatgttcacgttcagaggtcgatatcacg | |
| taaaataccgtctaaataagtaa | |
| gccatggggaggatgagggacgactgaatgcctgact | SEQ ID NO: 45 |
| ccaggtttgtggagatgtagtaattgctttccggtga | |
| agctttgt | |
| ata | |
| Cattcaggggtatggtatgtttaggatgttttcaagt | SEQ ID NO: 46 |
| tccccaaaccagatgttaagagaaataaaaatgtaaa | |
| gtataaacaatgcttaaaaaa | |
| caggagagaatcgacattgttggagtcaagtgtgtca | SEQ ID NO: 47 |
| ctgtataaggttggaaccctccctctcccacctaagg | |
| aagctcaggcagaggagaagggaaacagacaaatttc | |
| atgaaatcagccagg | |
| gtatgtggatgtcacctattctaatactacttgctgg | SEQ ID NO: 48 |
| aaagatcacctttcctctttgtaaatgatttgtggac | |
| tgtatttatgagggttcattactccattctgttctat | |
| tgatttgcttt | |

As mentioned, SEQ ID NOS: 1-29 are segments of genes encoding proteins whose functions are known, but which were not appreciated as being involved in neural plasticity prior to the present invention. Twelve of these proteins and their projected roles in neural plasticity are described in greater detail below.

Bach1:

SEQ ID NO:2 is a rat sequence that is homologous to a mouse sequence (GenBank Accession No. D-86603) encoding mouse Bach1. Bach1 is a ubiquitously expressed transcription factor expressed at high levels in the developing brain. Bach1 can bind to AP 1-like elements in DNA by itself, or associate with small Maf proteins (MafF, MafG, and MafK) to bind sequence specifically to DNA at sites referred to as MAREs (MAf Recognition Elements). It has been shown to play a role in globin expression and erythrocyte differentiation. The DNA binding activity of Bach1 is decreased by heme.

Bach1, together with its partner MafK, has been found to regulate gene expression in erythroid cell lines; however, there has been no evidence heretofore that Bach1 and MafK may also function as transcription factors in neurons. The inventors have determined that Bach1 is up-regulated in response to kainate treatment. In situ expression profiles revealed that Bach1 mRNA increases in granule cells of the dentate gyrus at about three hours after kainate treatment, then decreases after 10 hours post-kainate treatment. In CA1 pyramidal neurons, an increase in Bach1 mRNA is observed at about 10 hours post-kainate treatment.

It is known that AP-1, a sequence-specific transcriptional activator composed of members of the Jun and Fos families, which are also induced by seizures, can also bind to an element contained within the element (MAREs) recognized by Bach1/MafK. Moreover, MafK can also associate with c-Fos. Taken together, these observations indicate that MafK and Bach1 are components of an intricate network of transcription factors that regulate seizure-induced transcription of genes and therefore play a role in epilepsy. Bach1 and MafK appear to be seizure-induced transcription factors that play a role in cellular and functional alterations associated with TLE and neural plasticity.

Indeed, it has been determined that Bach1 can function as a transcriptional repressor. Bach1 acts coordinately and reciprocally with other transcription factors (AP-1) and CREB that are activators and are involved in synaptic plasticity.

Interferon Related Developmental Regulator (IFRD1/PC4):

SEQ ID NO:3 is the rat homolog of IFRD/PC4 (GenBank Accession No. J04511. PC4 cDNA encodes a 49 kDa protein that is homologous to beta-interferon and gamma-interferon. PC4 is an early NGF-inducible gene, which is transiently expressed during the in vitro differentiation of PC12 cells into a neuronal phenotype. PC12 cells are derived from a pheochromocytoma, which is an adrenal tumor, and can be driven into a neuronal phenotype by specific conditions. Immunofluorescence, electron microscopy, and subfractionation studies indicate the PC4 immunoreactivity is localized in the cytoplasm of PC12 cells, where it is increased transiently by NGF within 3 hours of treatment. Within 3 hours after addition of NGF, PC4 is also significantly expressed on the inner face of the plasma membrane, to which it is physically associated. After longer NGF treatment, PC4 disappears from the plasma membrane and appears in the nucleus, with reduced cytoplasmic expression. Localization in the nucleus is reversed by removal of NGF and closely parallels changes in the state of differentiation of the cell. The existence within the PC4 protein of a consensus sequence for the addition of myristic acid and of a putative sequence for the nuclear localization signal suggests possible mechanisms for the NGF dependent distribution. For an NGF-inducible IEG product, such growth factor-dependent localization of PC4 is a novel type of regulation in the pathways from the NGF receptor to the adjacent membrane proteins and to the nucleus. Thus, PC4 may have a role in signal transduction.

In situ hybridization analysis and other experiments have shown that PC4 is expressed at high levels along the whole neural tube of early rat embryos. PC4 mRNA expression is not uniform across the wall of the neural tube, but is most intense on the ventricular layer. At later stages, when the rate of proliferation and production of the postmitotic neurons decreases, PC4 gene expression also decreases and becomes restricted to the telencephalon, that is the last region to complete neurogenesis. The expression of the PC4 gene therefore appears to be correlated to the time span of proliferation of neuronal and glial precursors.

The inventors' discovery that expression of IFRD1/PC4, a growth factor inducible protein that influences proliferation and differentiation of neurons in early embryonic development, is also induced by seizures, indicates that this protein may play a role in circuit remodeling that includes neurogenesis, process outgrowth, and responses to alterations in neural connectivity as a consequence of seizures or other insults. Drugs that act on this target in adulthood could reduce unfavorable functional effects or promote desirable functional effects of circuit modification after injury.

Cis-Golgi SNARE:

SEQ ID NO:5 is the rat homolog of the Cis-Golgi SNARE p28 Sequence having GenBank Accession No. U49099. The p28 protein participates in the ER-Golgi transport of proteins, and the inventors have determined that expression of this gene is induced by kainate treatment in rats. As p28 is a protein that may effect ER-golgi transport of a variety of proteins, drugs acting on this seizure-induced protein target could have major effects on a variety of cellular processes. While permanent disruption of such processes would probably be undesirable or toxic, brief interruption after a brain insult could prevent functionally deleterious circuit modification that follows seizures or other injuries.

TFIID Subunit TAFII55:

SEQ ID NO:6 is a rat homolog of a mouse TAFII55) sequence having GenBank Accession No. NM 011901.1 Expressed from a gene located on mouse chromosome 5q31, TAFII55 is a part of the TFIID complex, which is composed of the TATA-binding protein (TBP) and approximately 10 TBP-associated factors (TAFs). TAFII55 interacts with TAFII250, hTAFII00, hTAFII28, hTAFII20, and hTAFII18, but not with hTAFII30 or TBP. Although hTAFII55 does not interact but itself with TBP, stable ternary complexes containing hTAFII55 and TBP can be formed in the presence of hTAFII250, hTAFII100, or hTAFII28. TAFII55 has been shown to interact with TAFII250 through its central region and with multiple activators, including Sp1, YY1, USF, CTF, adenoviral E1A, and human immunodeficiency virus type-1 Tat proteins through a distinct amino-terminal domain. The TAFII55-interacting region of Sp1 has been localized to its DNA-binding domain, which is distinct from the glutamine-rich activation domains previously shown to interact with Drosophila TAFII110.

As a component of the basal transcription apparatus, drugs acting on this seizure-induced protein target could have major effects on a variety of cellular processes. While permanent disruption of such processes might be undesirable, brief interruption of such processes after a brain insult could prevent functionally deleterious circuit modification that follows seizures or other injuries.

FGF-Inducible Gene 14 (FIN14):

SEQ ID NO:7 is a rat homolog of a mouse sequence having GenBank Accession No. U42386. Expression of the FIN genes is induced in response to FGF-4 as well as to serum in NIH3T3 cells with delayed kinetics, with maximum stimulation occurring 12-18 hours after growth factor treatment. Induction requires protein synthesis and is mostly transcriptional. FIN-12 is encoded by a broad range of previously described genes, some of which are proposed to have an important role in cell proliferation. The novel clones include an putative serine-threonine phosphatase (FIN13) and a gene with homology to NTP-binding proteins (FIN16).

Seizure-induced expression of FIN14, a growth factor inducible protein that influences proliferation and differentiation of neurons in early embryonic development, may play in role in circuit remodeling that includes neurogenesis, process outgrowth, and responses to alterations in neural connectivity as a consequence of seizures or other injurious insults. Drugs that act on this target in adulthood could alter unfavorable or promote desirable functional effects of circuit modification after injury.

Esterase-D or S-Formylglutathione Hydrolase:

SEQ ID NO:8 is a rat homolog of a mouse sequence having GenBank Accession No. M13450. Expressed from a gene located on human chromosome 13q14.1, S-formylglutathione hydrolase is an enzyme capable of hydrolyzing thiol esters of glutathione. The human esterase D gene has been localized cytogenically to the same sub-band of chromosome 13q14:11 as the retinoblastoma (RB) gene.

As a reducing agent that prevents formation of disulphide bonds, glutathione can exert effects on protein folding and degradation, and drugs targeting the hydrolysis of thiol esters of gluatthione might have effects on a variety of structural proteins or proteins involved in a range of cellular processes. As the redox state in a cell is dependent on acute conditions influenced by physiological processes, drugs acting on this target could have effects on both acute and chronic plasticity in neural circuits.

α-L-Fucosidase:

SEQ ID NO:9 is a rat homolog of sequence having GenBank Accession No. X16145. Expressed from a gene located on human chromosome 1p34 α-L-fucosidase is a lysosomal enzyme essential for the catabolism of oligosaccharides, glycolipids, and glycoproteins containing α-L-fucosides. Deficiency of this enzyme has been shown to be the cause of fucosidosis, which is an autosomal recessive disease. In neuronal cultures, fucosylation has been shown to influence process outgrowth and sprouting.

As fucosylation can influence process outgrowth, development of drugs that act on this target in adulthood could alter seizure-induced axon sprouting and prevent unfavorable functional effects of circuit modification after injury or proconvulsant effects of sprouting that worsens epilepsy.

Integrin α6 Subunit:

SEQ ID NO:12 is a rat homolog of sequence having GenBank Accession No. X69902. Laminin is the first extracellular matrix protein expressed in the developing mouse embryo, and is known to influence morphogenesis, cell migration, and polarization. Several laminin receptors are included in the integrin family of extracellular matrix receptors. Ligand binding by integrin heterodimers results in signal transduction events controlling cell motility. The integrin heterodimer α6:β1 is an important receptor for laminin in neurons, lymphocytes, macrophages, fibroblasts, platelets, and other cell types.

As an important molecule that influences axon guidance, Integrin 6 antagonist drugs could alter seizure-induced axon sprouting and prevent unfavorable functional effects of circuit modification after injury or proconvulsant effects of sprouting that worsens epilepsy.

Macrophilin (Microphilin and Actin Filament Crosslinker Protein) or ACF7:

SEQ ID NO:13 is a rat homolog of a human sequence having GenBank Accession No. AB007934. Expressed from a gene located on human chromosome 1p32, macrophilin (microphilin and actin filament crosslinker protein related to plectin and dystrophin, Accession # AB029290), appears to be a human homolog of the protein encoded by Drosophila gene kakapo, and shows a similarity to plectin and dystrophin. Comparison of the deduced protein sequences for macrophilin and kakapo reveals that they are 66% similar, and both have an NH₂-terminal actin-binding domain, a central rod region composed of spectrin-like repeats, and a COOH-terminal Gas2-related region. The predicted sequences of macrophilin are 5430 amino acids in length with a calculated molecular amass of 620 kDa, which is one of the largest sizes of identified human cytoskeletal proteins. High expression of macrophilin been observed in brain, heart lung, placenta, liver, kidney, and pancreas.

As a cytoskeletal protein, this protein may influence cellular morphology and thus could play a role in connectivity in neural circuits and seizure-induced plasticity. Drugs acting on this target could effect seizure-induced sprouting.

Synaptogyrin 3:

SEQ ID NO:15 is a rat homolog of a mouse sequence having GenBank Accession No. AK081164.1 Expressed from a gene located on human chromosome 16ptel, synaptogyrin 3 is and integral membrane protein associated with presynaptic vesicles in neuronal cells. Like other members of the synaptogyrin family, the predicted 206 amino acid SYNGR3 protein contains 4 transmembrane regions. Northern blot analysis revealed that the 2.2 kb SYNGYR3 mRNA is expressed in brain and placenta only.

A membrane protein associated with synaptic vesicles is potentially important for synaptic transmission, and drugs targeting this protein might have both acute and chronic effects on a wide range of neural circuits and brain functions.

Metallothionine I & II:

SEQ ID NOS:17 and 18 are rat homologs of a mouse sequence having GenBank Accession No. M11794. The metallothionine-1 and 2 (MT-1 and MT-2) genes comprise a multigene family whose expression is induced by zinc, cadmium, selenium, and cobalt, and whose protein products can chelate divalent metal ions.

Divalent cations such as Ca²⁺, Zn²⁺ and Mg²⁺ have prominent roles in receptor function and synaptic transmission, and potent effects on the function of neural circuits, including circuits of the hippocampus and dentate gyrus implicated in memory dysfunction and disorders such as epilepsy. Drugs that target metallothionines could have acute and chronic effects on hippocampal circuits and important brain functions.

GM3 Synthase or Sialytransferase 9:

SEQ ID NO:16 is a rat homolog of a sequence having GenBank Accession No. AB018049. Ganglioside GM3 is a major glycospingolipid in the plasma membrane and is widely distributed in vertebrates. GM3 synthase cDNA is 2359 kb, and encodes for a protein of 362 amino acids with a predicted molecular mass of 41.7 kDa. The gene is expressed in a tissue-specific manner, with predominant expression in brain, skeletal muscle, testis, and very low expression in liver.

This protein is an integral membrane component that is likely to be critical to most neurons, and is widely distributed in the brain. Drugs targeting this protein might have a diverse range of effects on functions of neural circuits.

B. Assays:

The above described genes and gene fragments, as well as their encoded proteins and fragments, may be used in a wide variety of diagnostic, prognostic and drug screening assays related to temporal lobe epilepsy and other plasticity-related disorders. These assays are based on the inventors' discovery that expression of these genes increases following seizure induction in association with a predictable sequence of evolving cellular alterations in neural circuits that are observed in a variety of neurological disorders initiated by neural injury. As a result, measurement of expression of any one of these genes, singly or in combination, provides a diagnostic or prognostic indicator of the seizure and other initial neural injuries that modify neural circuits.

Moreover, up-regulation of certain of the genes, but not others, is indicative of whether a seizure will or will not have long-term effects, e.g., will lead to increased propensity for spontaneous seizures. Accordingly, the inventors have devised assays that differentiate between seizures that are likely to lead to long-term effects and seizures not likely to have long-term effects. Specifically, through the use of the NMDA receptor inhibitor MK801, these assays differentiate between post-seizure NMDA receptor-mediated gene expression leading to long-term changes and modifications in neural circuitry associated with increased propensity for spontaneous seizures and adverse long-term functional effects, and post-seizure alterations in gene expression that is independent of NMDA receptor activity.

In certain embodiments of the invention, a seizure-induced change in expression of a single selected gene is measured. In preferred embodiments, expression of two or more genes is measured. Measurement of expression of a multiplicity of genes known to be affected by seizure yields additional information in the gene expression pattern. This observation has been extended through the use of oligonucleotide arrays representing all rat cDNA (Affymetrix, Inc.), providing a genomic pattern or “fingerprint” corresponding to the set or pattern of genes activated by the seizure. As a particularly significant example in accordance with the present invention, using the rat kindling model described herein, it is noted that the pattern of expression of gene fragments induced by seizures and detected by oligonucleotide arrays is different when NMDA receptor activity is inhibited by co-adminstration of MK801. Using the rat kindling model described herein, the inventors have identified approximately 200 genes that are differently regulated post-seizure if MK801 is administered to the animal subject at the time of seizure induction. Because MK801 blocks long-term consequences of repeated seizures including axon sprouting and functional kindling progression indicated by increasing susceptibility to seizures Sutula T, Koch J, Golarai G, Watanabe Y, McNamara J: J. Neurosci. 16(22):7398-7406, 1996), this modified differential expression pattern detected when a seizure is induced during administration of MK801 is of utility as a genomic “fingerprint’ of the set of genes underlying long-term effects of seizure. This genomic “fingerprint” may be used as a method or tool to identify therapeutic interventions, including but not limited to administration of compounds or other manipulations of neural circuitry such as stimulation delivered to regions of neural circuitry, which modify the long-term effects of seizures or other related conditions characterized by neuronal injury and sprouting.

In various embodiments of the present invention, changes in gene expression are measured in one or both of two ways: (1) measuring transcription through detection of mRNA produced by a particular gene; and (2) measuring translation through detection of protein produced by a particular transcript.

Decreased or increased expression can be measured at the RNA level using any of the methods well known in the art for the quantitation of polynucleotides, such as, for example, PCR, RT-PCR, RNase protection, Northern blotting and other hybridization methods. The genes that are assayed or interrogated according to the present invention are typically in the form of mRNA or reverse transcribed mRNA. The genes may be cloned and/or amplified. The cloning itself does not appear to bias the representation of genes within a population. However, it may be preferable to use polyA+ RNA as a source, as it can be used with fewer processing steps.

Oligonucleotide or polynucleotide probes for interrogating the tissue or cell sample are prepared using the sequence information set forth herein for the 48 isolated gene fragments, including genes and ESTs present in public databases as also set forth herein. The probes should be of sufficient length to specifically hybridize substantially exclusively with appropriate complementary genes or transcripts. Typically, the oligonucleotide probes will be at least 10, 12, 14, 16, 18, 20 or 25 nucleotides in length. In some cases, longer probes of at least 30, 40, 50, 60, 70, 80, 90 or 100 nucleotides are desirable, though probes longer than 100 nucleotides may be suitable in some embodiments.

In preferred embodiments of the present invention, immobilized nucleic acid probes are used for the rapid and specific detection of nucleic acid molecules and their expression patterns. Typically, a nucleic acid probe is linked to a solid support and a target nucleic acid (e.g., a genomic nucleic acid, an amplicon, or, most commonly, an amplified mixture) is hybridized to the probe. Either the probe, or the target, or both, can be labeled, typically with a fluorophore or other tag, such as streptavidin. Where the target is labeled, hybridization is detected by detecting bound fluorescence. Where the probe is labeled, hybridization is typically detected by quenching of the label. Where both the probe and the target are labeled, detection of hybridization is typically performed by monitoring a color shift resulting from proximity of the two bound labels. A variety of labeling strategies, labels, and the like, particularly for fluorescent based applications are known in the art.

Assay techniques that can be used to determine levels of a protein in a sample are also well known to those of skill in the art. Such assay methods include radioimmunoassays, competitive-binding assays, Western blot analysis and ELISA assays. In the assay methods utilizing antibodies, both polyclonal and monoclonal antibodies are suitable for use in the present invention. Such antibodies may be immunologically specific for a particular protein, or an epitope of the protein, or a protein fragment, as would be well understood by those of skill in the art. Methods of making polyclonal and monoclonal antibodies immunologically specific for a protein or peptide are also well known in the art.

Preferred embodiments of the present invention utilize immobilized antibodies for the detection and quantification of proteins produced by the seizure-induced genes described herein. Though proteins may be detected by immunoprecipitation, affinity separation, Western blot analysis and the like, a preferred method utilizes ELISA-type methodology wherein the antibody is immobilized on a solid support and a target protein or peptide is exposed to the immobilized antibody. Either the probe, or the target, or both, can be labeled. A variety of labeling strategies, labels, and the like, are known in the art.

In particularly preferred embodiments of the invention, expression patterns or profiles of a plurality of seizure-induced genes are observed utilizing arrays of probes for detecting target nucleic acids or proteins. In one embodiment, arrays of oligonucleotide or polynucleotide probes are utilized, whereas another embodiment utilizes arrays of antibodies or other proteins that bind specifically to the seizure-induced gene products. Such arrays are commercially available (e.g. through Affymetrix, Inc., Applied Biosystems, Inc., Agilent, Inc.), or they may be custom made according to known methods, such as in-situ synthesis on a solid support or attachment of pre-synthesized probes to a solid support via micro-printing techniques. In preferred embodiments, arrays of oligonucleotide or antibody probes are custom made to specifically detect transcripts or proteins produced by several or all of the 48 seizure-induced genes or gene fragments described above.

In one aspect of the invention, assays are provided for identifying new drugs for treatment of TLE and other seizure-related disorders. Several types of assays are featured. One embodiment features high throughput screening assays utilizing the genes and encoded proteins with known function, which the inventors have now identified through the kainic acid or kindling model as being seizure-induced. As mentioned above, these genes include the following: Bach1, Interferon Related Developmental Regulator (IFRD1/PC4), Cis-Golgi SNARE p28, TFIID subunit TAFII55, FGF-Inducible gene (FIN14), Esterase-D or S-formylglutathione hydrolase, -L-fucosidase, Integrin 6 subunit, Macrophilin (microphilin and actin filament crosslinker protein or ACF7), Synaptogyrin 3, Metallothionine I and II, and GM3 synthase (or Sialytransferase 9).

One type of assay involves measuring the activity of the protein encoded by one of the aforementioned in the presence or absence of a candidate drug. Such activity assays are well known in the art. If a cell-free activity assay is available for the selected protein, such an assay is simply conducted on the purified protein in the presence or absence of the test compound. Candidate drugs are selected based on their ability to positively or negatively regulate activity of the purified protein. As one example, a candidate drug may be tested for its ability to inhibit binding of Bach1 to its DNA recognition sequence in a gene promoter. Assays of this type generally comprise contacting a chimeric construct, comprising a reporter gene controlled by the promoter containing the DNA recognition sequence, with the DNA binding protein (e.g., Bach1 or Bach1/MafK) in the presence or absence of the test compound. It should be noted that assays of this type are usually performed in a recombinant cellular system, as described below, but they can also be performed in a cell-free system in some instances.

For such in vitro activity assays, it is desirable to have a source of the purified protein of interest. One or more of the protein products of the genes mentioned above may be commercially available, or purifiable in significant quantities from an appropriate biological source, e.g., cultured cells. Alternatively, the proteins may be recombinantly produced from an isolated gene or cDNA by expression in a suitable procaryotic or eucaryotic expression system, and thereafter purified, as is also well known in the art.

Another embodiment of the invention comprises in vitro cellular assays for expression of seizure-induced genes or activity of their encoded proteins. For these embodiments, a nucleic acid construct comprising a seizure-induced gene of the invention is introduced into host cells. In a preferred embodiment, mammalian cell lines are utilized. Host cells contemplated for use include, but are not limited to NIH3T3, CHO, HELA, PC12, as well as non-mammalian cells such as yeast, bacteria and insect cells. The coding sequences are operably linked to appropriate regulatory expression elements suitable for the particular host cell to be utilized. Methods for introducing nucleic acids into host cells are well known in the art. Such methods include, but are not limited to, transfection, transformation, calcium phosphate precipitation, electroporation and lipofection.

The recombinant cells are used to identify compounds which modulate expression of the seizure-induced genes or activity of their encoded proteins. Modulation of bach1 activity, for example, may be assessed by measuring alterations in bach1 DNA binding activities in the presence of the test compound. Such assays preferably comprise a system in which the promoter comprising the Bach1 DNA recognition sequence is operably linked to a reporter gene. Alternatively, disruption of Bach1/Maf K protein-protein interactions mediated by a test compound can be assessed.

For gene expression assays, it is preferred to prepare an artificial construct comprising the promoter of a selected seizure-induce gene, such as Bach1, operably linked to a reporter gene. The reporter construct is introduced a cultured cell, such as the standard host cell lines described above, or cell lines of primary neuronal or glial cells obtained from different regions of the nervous system. The assay is performed by monitoring expression of the reporter gene in the presence or absence of the test compound. Candidate drugs are selected based on their ability to positively or negatively affect expression of the gene.

Another in vitro assay of the present invention involves treating cultured neural cells with kainic acid and observing the change in expression of one or more of the seizure-induced genes or gene fragments represented by SEQ ID NOS: 1-48 (or expanded to include all expressed genes in a cell), as compared with cells not exposed to kainic acid.

The DNA or antibody array technology described above is particularly useful for this embodiment, though not required. In these assays, a suitable cell line is treated with kainic acid in the presence or absence of a test compound, and expression of one or more selected genes, or the expression profile of many or all of the seizure-induced genes or gene fragments described herein, is measured and differences noted. In a variation of this assay, the selected cell line contains NMDA receptors and the assay is conducted in the presence or absence of MK801 as well as in the presence or absence of the test compound. Such an assay will yield information as to whether the test-compound mediates a change in NMDA receptor-dependent or -independent gene expression, which is indicative of the compound's potential efficacy in treating long-term versus short-term effects of seizures or initial neural injuries.

In further embodiments of the invention, in vivo or in situ assays are provided. These assays are particularly suitable for further testing of candidate drugs identified via the high-throughput screening assays described above. Typically, assays of this type involve inducing seizures in animals in the presence or absence of the test compound, then monitoring changes in expression of one or more selected genes, or a change in the expression profile of many or all of the 48 seizure-induced genes described herein, or expanded to all expressed genes in the animal subject, e.g., through the use of commercially available expressed gene arrays, preferably using DNA or antibody arrays. Further, the gene expression results may be correlated with other physiological or biochemical indications of long-term effects of the seizures, such as mossy fiber sprouting.

An exemplary animal model system for assays of the present invention is the kindling method in rats, wherein repeated activation of neural pathways, either by electrical stimulation or by repeated chemical activation of neural pathways, is used to induce increasing susceptibility to evoked seizures and eventual progression to spontaneous seizures, as described hereinabove. Methods for performing kindling assays in rats are described in the examples. For electrical induction, rats are anesthetized and stereotaxically implanted with chronic electrodes for stimulation and recording in the angular bundle or in other pathways of the central nervous system. After a two-week recovery period, the afterdischarge (AD) threshold is determined. Expression of selected genes or gene expression profiles may be measured at periodic intervals (e.g., 1, 3, 7, 10, and 24 hours) after the initial AD and at additional time points to define the time course of expression. Thereafter, rats receive kindling stimulation twice daily (5 days per week), and gene expression is assessed at periodic time points after one or more Class V seizures. An alternative method for inducing seizures is chemical induction by intraperitoneal administration of kainic acid at 9-12 mg/kg, which produces status epilepticus followed by gradual progression to spontaneous seizures, with subsequent monitoring of gene expression at time intervals after induction of status epilepticus.

In a variation of the above-described in vivo assay, seizures may be induced in an animal the presence or absence of MK801 as well as in the presence or absence of the test compound. Similar to the cultured cell assay described above, this type of assay will yield information as to whether the test-compound mediates a change in NMDA receptor-dependent or -independent gene expression, which is indicative of the compound's potential efficacy in treating seizure-related disorders, i.e., conditions that lead to increased or spontaneous seizures versus those that do not, or other conditions in which evolving plasticity-related alterations in neural circuits follow initial neuronal injury. Assays of this type are further informative because MK801 treatment renders animals “slow kindling,” i.e., resistant to increased susceptibility of evoked seizures and spontaneous seizures following a course of repeated neural activation, and resistant to evolving neural circuit changes or plasticity following initial neuronal injury. Comparative use of “slow kindling” and “fast kindling” (animals not treated with MK801) in evaluating a candidate drug can yield valuable in vivo information as to the efficacy of that drug on NMDA receptor-dependent, versus—independent gene expression. The use of MK801 in a rat kindling model is described in detail by Sutula et al. (J. Neurosci. 16: 7398-7406, 1996). The methods described therein are appropriate for use in the present invention. As an additional advantage to the use of MK801 in assays of the invention, such assays may be useful in identifying compounds that have an effect similar to that of MK801, i.e., compounds that can inhibit the progression of kindling. Such compounds are expected to be particularly critical for the prevention of recurring or spontaneous seizures, after an initial seizure has been experienced, or in favorably modifying long-term consequences of neuronal injury in other conditions such as stroke, trauma, neurodegenerative diseases in which plasticity contributes to adverse function or outcome.

The NMDA antagonist MK801, available from commercial sources including Sigma Chemical Co. (St. Louis, Mo.), is exemplified for use herein because it can be administered at doses that impede the progression of kindling without having an antiseizure effect. It will be understood by those of skill in the art that any NMDA antagonist having this feature will be suitable for use in the present invention. Other NMDA antagonists that may be used include CPP-ene, among others.

Also contemplated for use in the present invention are experimental animals that have been bred or genetically altered to exhibit slow and fast kindling upon appropriate electrical stimulation. Use of such animals in any of the above-described in vivo assays will provide information regarding candidate drugs that is of particular relevance to the use of those drugs in humans. Further, transgenic animals have specific alterations, such as “knockout” mice defective in one of the genes described herein, will be of utility for analysis of candidate drugs against a specific target. Methods of introducing transgenes and knockouts in laboratory animals, particularly mice and rats, are known to those of skill in the art. Three common methods include: (1) integration of retroviral vectors encoding the foreign gene of interest into an early embryo; (2) injection of DNA into the pronucleus of a newly fertilized egg; and (3) the incorporation of genetically manipulated embryonic stem cells into an early embryo.

The in vivo methods described above are not only useful for screening candidate anti-epileptic drugs, they are also useful for establishing diagnostic and prognostic indicators of whether a patient has a disorder of a type that may lead to increased or spontaneous seizures, or other unfavorable functional outcomes after neuronal injury as a consequence of plasticity-related circuit alterations. In particular, using NMDA agonists such as MK801, the aforementioned methods will establish gene expression profiles that are indicative of either (1) NMDA receptor-mediated downstream events (known to lead to increased or spontaneous seizures or adverse long-term outcomes), or (2) NMDA receptor-independent events. After such respective profiles are generated, individual genes that appear to be uniquely associated with one profile but not the other may be isolated and further characterized for their roles in seizure-induced physiology.

Thus, further embodiments of the present invention relate to diagnostic and prognostic methods for use with patients in connection with seizure-related disorders or other conditions in which there is initial neural injury followed by evolving long-term effects, as in stroke, trauma and various neurodegenerative diseases. As used herein, the term “patient” refers to humans or animals, inasmuch as the present invention is anticipated to have utility in human and veterinary medicine.

A typical medical or veterinary diagnostic test will comprise obtaining a sample of cells or tissue from the patient in which a seizure-related or injury-induced change in gene expression is expected to occur. Such cells or tissues include, but are not limited to blood, skin, and cerebrospinal fluid. The sample is then analyzed for either (1) increased expression of one or more selected genes, via detection of mRNA or protein, or (2) a particular gene expression profile, e.g., via gene or protein array technology, as described in detail above. Such a diagnostic procedure should lead to a determination of the nature of the seizure or other disorder, with respect to the chances that the patient will experience recurring seizures, or other adverse functional outcomes after initial neural injury.

The aforementioned diagnostic procedure may also be extended to provide prognostic information for a patient's recovery from one of the disorders mentioned above, or to monitor a patient's progress in response to a therapeutic regimen. In these situations, the diagnostic assay is performed at intervals during the patient's recovery or course of treatment after seizure or initial injury, and a change in expression of a target gene, or a particular change in the pattern of gene expression, is indicative of the patient's level of recovery or improvement.

Another aspect of the invention features compositions of matter to facilitate practice of the assays described above. These compositions comprise collections of two or more probes or primers for use in detecting the seizure-induced, plasticity-related genes, gene fragments and encoded proteins of the invention. In one embodiment, the compositions comprise collections of two or more oligonucleotides or polynucleotides that specifically hybridize with a nucleic acid molecule selected from SEQ ID NOS: 1-48. The collection may comprise a primer pair for amplifying the sequence. Preferably, the collection comprises two or more probes for detecting two or more of SEQ ID NOS: 1-48, respectively. In a preferred embodiment, the collection comprises a larger plurality of probes, e.g., 5, 10, 15, 20, 25 or more probes, each of which hybridizes specifically with part or all of one of the sequences of SEQ ID NOS: 1-48. In a preferred embodiment, the oligonucleotide probes are immobilized on a solid support. In a particularly preferred embodiment, they are immobilized in an array format, most preferably in a miniature or micro-array. Such micro-arrays are known in the art, and are sometimes referred to as “DNA chips”, “microchips”, “biological chips” and other similar terms, and may contain the entire array of cDNAs altered by seizures, in addition to SEQ ID NOS: 1-48.

In another embodiment, these compositions comprise two or more protein binding substances capable of specifically binding proteins or protein fragments encoded by the genes and gene fragments of SEQ ID NOS: 1-48. Such binding substances may be any molecule to which the protein or peptide specifically binds, including DNA (for DNA binding proteins), antibodies, cell membrane receptors, peptides, cofactors, lectins, sugars, polysaccharides, cells, cell membranes, organelles and organellar membranes. In a preferred embodiment the binding substances are antibodies and the collection comprises two or more antibodies for detecting two or more proteins or peptides encoded by SEQ ID NOS: 1-48, respectively. In a preferred embodiment, the collection comprises a larger plurality of antibodies, e.g., 5, 10, 15, 20, 25 or more, each of which binds immunospecifically with part or all of a protein or peptide encoded by one of the sequences of SEQ ID NOS: 1-48. In a preferred embodiment, the antibodies are immobilized on a solid support. In a particularly preferred embodiment, they are immobilized in an array format, most preferably in a miniature or micro-array, as described above for oligonucleotide probes, and may contain the entire array of proteins altered by seizures, in addition to SEQ ID NOS: 1-48.

Still another aspect of the invention features test kits for use in one or more of the in vitro, in vivo and medical/veterinary diagnostic assays described above. One type of kit comprises one or more pairs of primers for amplifying nucleic acids corresponding to the seizure-induced genes and gene fragments described herein. The kit may further comprise samples of total mRNA derived from tissue of various physiological states, for use as controls. The kit may also comprise buffers, nucleotide bases, and other compositions to be used in hybridization and/or amplification reactions. Each solution or composition may be contained in a vial or bottle and all vials held in close confinement in a box for commercial sale.

Another type of kit comprises one or more oligonucleotide or antibody probes, wherein the oligonucleotide probe hybridizes specifically with a seizure-induced, plasticity-related gene or gene fragment of the invention, or the antibody is immunologically specific for a protein encoded by the seizure-induced gene or gene fragment. In preferred embodiments, the oligonucleotide or antibody probes are immobilized on a solid support. In a particularly preferred embodiment, the kit comprises immobilized arrays of oligonucleotide or antibody probes, the arrays being composed of probes specific for each of the seizure-induced genes or gene fragments of the invention, or proteins encoded thereby. These kits also may contain appropriate control samples of mRNA or protein from tissues of known physiological state, to be used as controls in the assays. They may further comprise buffers and reagents for performing the assays. Each solution, reagent or composition in the kit may be contained in a vial or bottle and all vials held in close confinement in a box for commercial sale.

The following examples are provided to describe the invention in greater detail. They are intended to illustrate, not to limit, the invention.

### EXAMPLE 1

Preparation of RNA from Seizure-Induced and Control Rats. For chemical induction of seizures, the rats were injected intraperitoneally with kainic acid at 9-12 mg/kg. The rats were killed after 3 hours, and the hippocampus was removed and rapidly frozen on dry ice. Total RNA was extracted and isolated with the RNeasy kit (Qiagen), and from control rats. For induction of seizures by kindling, adult male Sprague Dawley rats (250-300 gm) were anesthetized with a combination of ketamine 80 mg/kg IM and xylazine 10 mg/kg IM, and stereotaxically implanted with chronic electrodes for stimulation and recording in the angular bundle (8.1 mm posterior, 4.4 mm lateral, and 3.5 mm ventral to bregma). The electrodes were fixed to the skull with screws and dental acrylic. After a two-week recovery period, the after discharge or evolved seizure threshold (AD) was determined by a standard procedure (Cavazos et al., J. Neurosci. 14: 3106-3121, 1994).

### EXAMPLE 2

Differential Display PCR Procedure. Differential display PCR (DD-PCR) was carried out on total RNA extracted from rat brain at 3 hours after seizures induced by kainic acid using the GeneHunter kit according to manufacturer's protocol. Briefly, 50 μg of RNA were treated with RNase-free DNase I (Promega) for 30 min at 37° C. and RNA quality and yield was verified on a 1.2% formaldehyde agarose gel. First-strand cDNA was synthesized using 0.5 μg total RNA as template and the appropriate anchor primer, using Superscript preamplification kit (Invitrogen). DD-PCR was carried out in 20 μl total volume, and included 0.8 μM of one of the three 3′ anchor primers, and one of the eight arbitrary 5′ primers, 2.5 μM dNTP, 2.5 mM MgCl2, 2 μCi [α-³²P]dATP (3000 Ci/mM; NEN), 2 μl cDNA prepared from 50 ng total RNA, and 2.5 units of Taq DNA polymerase. The DD-PCR reaction was started with a 3-min incubation at 94° C., followed with 40 cycles of 30 sec at 94° C., 2 min at 40° C. and 30 sec at 72° C., and a final incubation of 5 min at 72° C. Then, 10 μl of the reaction mixture were mixed with 10 μl of 95% formamide, containing 0.05% bromophenyl blue and 0.05% xylene cyanol, incubated for 5 min at 75° C. and 6 μl were analyzed on a 6% DNA sequencing gel. The gel was dried and exposed to an X-ray film (Kodak), and DNA bands expressed differentially in the kainite treated animals were identified, cut from the gel, and soaked in 100 μl dH₂O for at least 12 hours. Subsequently, samples were boiled for 15 min, centrifuged at 10000×g, and 10 μl of the supernatant were employed as a template to re-amplify the band of interest, with the same set of primers that produced the original band and using the same PCR conditions described for DD-PCR, except that 25 μM dNTP and no isotope were used. Ten μl of the PCR reaction were run on a 1.2% agarose gel. Bands with predicted sizes were excised, purified with the DNA extraction kit (Qiagen), and cloned into pGEM-T Easy Vector (Promega). Plasmid DNAs were purified with Plasmid Mini Kit (Qiagen) and those harboring inserts were sequenced with forward and reverse sequencing primers. The obtained sequence data were then compared with the information in the GeneBank (NCBI) database.

### EXAMPLE 3

Array Hybridization Procedure. Gene expression was evaluated by array hybridization techniques at 3 hours after an evoked kindled seizure, and at 3 hours after an evoked kindled seizure in a rat that received the NMDA antagonist MK801 (0.5 mg/kg IP) 30 minutes before the kindling stimulation. For double-stranded cDNA synthesis, 50 μg of RNA were treated with RNase-free DNase I (Promega) for 30 min at 37° C. and RNA quality and yield was verified on a 1.2% formaldehyde agarose gel. First-strand cDNA was synthesized using 0.5 μg total RNA as template and the appropriate anchor primer, using Superscript preamplification kit (Invitrogen). Second-strand cDNA was synthesized from the product of the first-strand synthesis, using the Superscript system (Invitrogen), and subjected to cleanup in accordance with the manufacturer's instructions. The Enzo® Bioarray High-Yield RNA Transcript Labeling Kit (supplied by Affymetrix, Inc.) was used for generating cRNA targets, in accordance with the manufacturer's instructions. The cRNA was fragmented in preparation for hybridization onto GeneChip® Rat Expression Set 230 probe arrays (Affymetrix, Inc., Santa Clara, Calif.). An aliquot of the fragmented cRNA was utilized for hybrizidation onto the probe array for 16 hours, in accordance with manufacturer's instructions. Following hybridization, arrays were washed, stained and scanned. Results were analyzed with the GeneChip® analytical software.

Results are shown in the attached Table. Decreased expreassion is indicated with “D,” while increased expression is indicated with “I.”

| | | 081902B | |
|---|---|---|---|
| | 081902B | vs | |
| | vs | 082102A | |
| | 082102A | Change | |
| Array Designation | Change | p-value | Descriptions |
| E03229cds_s_at | D | 1 | cDNA encoding rat liver L-cysteine oxygen oxidoreductase |
| L17127_g_at | D | 0.999966 | L17127 RATRN3 Rattus norvegicus proteasome RN3 |
| | | | subunit mRNA |
| AF007107_s_at | D | 0.999983 | AF007107 Rattus norvegicus soluble cytochrome b5 |
| | | | mRNA |
| M22756_at | D | 0.999926 | M22756 Rat 24-kDa subunit of mitochondrial NADH |
| | | | dehydrogenase mRNA |
| rc_AI137856_s_at | D | 0.998434 | |
| rc_AI070108_at | D | 0.99996 | |
| rc_AA799499_at | D | 0.999885 | |
| rc_AI177256_at | D | 0.999104 | |
| rc_AI102868_g_at | D | 0.999867 | |
| rc_AI103396_g_at | D | 1 | |
| rc_AI104077_at | D | 0.998848 | |
| rc_AI171355_s_at | D | 1 | |
| rc_AI179150_s_at | D | 1 | |
| rc_AA892248_at | D | 0.999996 | |
| rc_AA942685_at | D | 0.999995 | |
| rc_AA799889_at | D | 0.999963 | |
| rc_AI231778_at | D | 0.999563 | |
| rc_AI169265_at | D | 1 | |
| rc_AI171243_at | D | 0.999563 | |
| AF048687_s_at | D | 0.999988 | AF048687 Rattus norvegicus UDP-Gal: glucosylceramide |
| | | | beta-1,4-galactosyltransferase |
| AF067727_s_at | D | 0.997888 | AF067727 Rattus norvegicus Smad1 protein (Smad1) |
| | | | mRNA |
| AF071225_at | D | 0.999954 | AF071225 Rattus norvegicus cyclophilin B mRNA, |
| D44495_s_at | D | 0.999867 | D44495 RATAPEXN Rat adult brain mRNA for APEX |
| | | | nuclease |
| J01435cds#8_s_at | D | 0.998983 | J01435cds#8 RATMTCYOS Rattus norvegicus |
| | | | mitochondrial cytochrome oxidase subunits |
| L32591mRNA_at | D | 0.999689 | L32591mRNA RATGADD45X Rattus norvegicus |
| | | | GADD45 mRNA |
| M27467_at | D | 0.999998 | M27467 RATCOXHRT Rattus norvegicus heart |
| | | | cytochrome oxidase subunit VIc (COX-VIc) mRNA, |
| M28255_s_at | D | 0.999822 | M28255 RATCYO8A Rat cytochrome c oxidase subunit |
| | | | VIII mRNA |
| M28648_s_at | D | 0.99995 | M28648 RATNALPH2 Rattus norvegicus Na, K-ATPase |
| | | | alpha-2 subunit mRNA |
| M91597_s_at | D | 1 | M91597 RATRBLNDP Sprague-Dawley (p18-12d) RBL- |
| | | | NDP kinase 18 kDa subunit (p18) mRNA |
| rc_AA926137_s_at | D | 0.999997 | |
| D17309_at | D | 0.998009 | D17309 Rat mRNA for delta-4-3-ketosteroid 5-beta- |
| | | | reductase |
| D84450_at | D | 0.998613 | D84450 Rat mRNA for Na+, K+-ATPase beta-3 subunit |
| U38801_at | D | 0.998233 | U38801 Rattus norvegicus high molecular weight DNA |
| | | | polymerase beta (rnpolb) mRNA |
| V01217_at | D | 0.999987 | V01217 Rat gene encoding cytoplasmic beta-actin |
| X72757_g_at | D | 0.999987 | X72757 R. norvegicus cox VIa gene (liver) |
| X70369_s_at | D | 0.99996 | X70369 R. norvegicus mRNA for pro alpha 1 collagen type |
| | | | III |
| X53427_at | D | 0.998613 | X53427 Rat mRNA for glycogen synthase kinase 3 alpha |
| U64030_at | D | 0.99971 | U64030 Rattus norvegicus dUTPase mRNA |
| U69109_s_at | D | 0.999981 | U69109 Rattus norvegicus calcium-dependent tyrosine |
| | | | kinase mRNA |
| D00569_at | D | 0.997888 | D00569 Rat mRNA for 2,4-dienoyl-CoA reductase |
| X62952_at | D | 1 | X62952 R. norvegicus mRNA for vimentin |
| J02720_at | D | 0.999667 | J02720 Rat liver arginase mRNA |
| K01932_f_at | D | 0.999835 | K01932 Rat liver glutathione S-transferase Yc subunit |
| | | | mRNA |
| U36992_at | D | 0.999921 | U36992 RRU36992 Rattus rattus cytochrome P450 |
| | | | Cyp7b1 mRNA |
| U86635_at | D | 0.999966 | U86635 RNU86635 Rattus norvegicus glutathione s- |
| | | | transferase M5 mRNA |
| U86635_g_at | D | 0.999999 | U86635 RNU86635 Rattus norvegicus glutathione s- |
| | | | transferase M5 mRNA |
| X59051cds_s_at | D | 0.999999 | X59051cds RNRPS29 R. norvegicus gene for ribosomal |
| | | | protein S29 |
| Y12517cds_at | D | 0.999729 | Y12517cds RNOMB5MIT R. norvegicus mRNA for |
| | | | mitochondrial isoform of cytochrome b5 |
| rc_AA925717_at | D | 0.999983 | rc_AA925717 UI-R-A1-ec-e-06-0-UI.s1 UI-R-A1 Rattus |
| | | | norvegicus cDNA clone similar to Mus musculus apoptosis |
| | | | associated tyrosine kinase |
| A09811cds_s_at | D | 0.999991 | A09811cds R. norvegicus mRNA for BRL-3A binding |
| | | | protein |
| AB008807_at | D | 0.999981 | AB008807 Rattus rattus mRNA for glutathione-dependent |
| | | | dehydroascorbate reductase |
| AF028784cds#1_s_at | D | 0.999991 | AF028784cds#1 Rattus norvegicus glial fibrillary acidic |
| | | | protein alpha (GFAP) gene |
| AF028784mRNA#1_{—} | D | 0.999876 | AF028784mRNA#1 Rattus norvegicus glial fibrillary acidic |
| s_at | | | protein alpha (GFAP) gene |
| AF035953_at | D | 0.999915 | AF035953 Rattus norvegicus kinesin-related protein KRP4 |
| | | | (KRP4) mRNA |
| AF036548_at | D | 0.99978 | AF036548 Rattus norvegicus RGC-32 (RGC-32) mRNA |
| AF059678_s_at | D | 0.999643 | AF059678 Rattus norvegicus VIP-receptor-gene repressor |
| | | | protein mRNA |
| AF060173_at | D | 0.999765 | AF060173 Rattus norvegicus SV2 related protein (SVOP) |
| | | | mRNA |
| AF063890_s_at | D | 0.999667 | AF063890 Rattus rattus focal adhesion kinase-related |
| | | | protein (PYK2) mRNA |
| AF064868_at | D | 0.999809 | AF064868 Rattus norvegicus brain-enriched guanylate |
| | | | kinase-associated protein 1 mRNA |
| AF068860_s_at | D | 0.99943 | AF068860 Rattus norvegicus beta defensin-1 mRNA |
| AF074609mRNA_f_{—} | D | 0.998233 | AF074609mRNA Rattus norvegicus MHC class I antigen |
| at | | | (RT1.EC3) gene |
| AF083269_g_at | D | 0.999915 | AF083269 Rattus norvegicus p41-Arc mRNA, complete |
| | | | cds |
| AF087696_at | D | 0.999729 | AF087696 Rattus norvegicus dlg 2 mRNA, partial cds |
| D21869_s_at | D | 0.999729 | D21869 RATPFKM04 Rat mRNA for PKF-M |
| | | | (phosphofructokinase-M) |
| D26154cds_at | D | 0.999991 | D26154cds RATRB109 Rat mRNA for RB109 (brain |
| | | | specific protein) |
| D82074_g_at | D | 1 | D82074 RATBHF1MA Rattus sp. mRNA for BHF-1 |
| D89730_at | D | 0.998774 | D89730 Rattus rattus T16 mRNA |
| D89730_g_at | D | 0.999104 | D89730 Rattus rattus T16 mRNA |
| J01435cds#1_s_at | D | 0.999971 | J01435cds#1 RATMTCYOS Rattus norvegicus |
| | | | mitochondrial cytochrome oxidase subunits I, II, III genes, |
| | | | ATPase subunit 6 gene |
| J01435cds#4_s_at | D | 0.999999 | J01435cds#4 RATMTCYOS Rattus norvegicus |
| | | | mitochondrial cytochrome oxidase subunits I, II, III genes, |
| | | | ATPase subunit 6 gene |
| L13635_s_at | D | 0.999996 | L13635 RATHRS Rattus rattus insulin-induced growth- |
| | | | respons protein (HRS) mRNA |
| L26292_g_at | D | 0.999987 | L26292 RATFSHREG Rattus norvegicus (clone 59) FSH- |
| | | | regulated protein mRNA |
| M10934_s_at | D | 0.999996 | M10934 RATRBPA Rat retinol-binding protein (RBP) |
| | | | mRNA |
| M16960_s_at | D | 0.999941 | M16960 RATPK2CCD Rat calcium-calmodulin-dependent |
| | | | protein kinase II mRNA |
| M27207mRNA_s_at | D | 1 | M27207mRNA RATCOL1A1 Rattus norvegicus (clone |
| | | | pL6-3-1) alpha-1 type I collagen mRNA |
| M29866_s_at | D | 0.999997 | M29866 RATC3 Rat complement component C3 mRNA |
| M89646_g_at | D | 0.999999 | M89646 RATRPRS24A Rattus norvegicus ribosomal |
| | | | protein S24 mRNA sequence |
| M91595exon_s_at | D | 1 | M91595exon RATILGFBPA Rattus norvegicus insulin-like |
| | | | growth factor binding protein-2 gene |
| M94557_s_at | D | 0.999987 | M94557 RATMTSSB Rat specific single stranded DNA |
| | | | binding protein mRNA |
| M94918mRNA_f_at | D | 1 | M94918mRNA RATBETGLOX Rat beta-globin gene |
| M94919mRNA_f_at | D | 1 | M94919mRNA RATBETGLOY Rat beta-globin gene |
| S56141_s_at | D | 0.999211 | S56141 sodium-dependent neurotransmitter transporter |
| S75275_at | D | 0.998009 | S75275 RVLG = vasa-like gene protein mRNA |
| S75687_s_at | D | 0.999926 | S75687 glutamate/aspartate transporter mRNA |
| S76742_at | D | 0.999999 | S76742 neurotransmitter transporter rB21 mRNA |
| S76779_s_at | D | 0.999809 | S76779 rApoE = apolipoprotein E mRNA |
| S78218_g_at | D | 1 | S78218 protein phosphatase 1 beta mRNA |
| S79304_s_at | D | 1 | S79304 cytochrome oxidase subunit I, Ser-tRNA [rats, |
| | | | Sertoli cells, mRNA Mitochondrial |
| U10894_s_at | D | 0.999689 | U10894 RNU10894 Rattus norvegicus mRNA expressed in |
| | | | carotid artery tissue |
| U31160mRNA_s_at | D | 0.99943 | U31160mRNA RNCR1601 Rattus norvegicus SH3 domain |
| | | | binding protein (CR16) gene |
| U36771_at | D | 0.99776 | U36771 RNU36771 Rattus norvegicus glycerol 3- |
| | | | phosphate acyltransferase mRNA, nuclear gene encoding |
| | | | mitochondrial protein |
| U67207_s_at | D | 0.998124 | U67207 RNU67207 Rattus norvegicus leptin receptor (OB- |
| | | | R) mRNA |
| U75404UTR#1_s_at | D | 0.999999 | U75404UTR#1 RNU75404 Rattus norvegicus Ssecks 322 |
| | | | mRNA |
| U75405UTR#1_f_at | D | 0.999998 | U75405UTR#1 RNU75405 Rattus norvegicus alpha 1 type |
| | | | I collagen mRNA |
| U95001UTR#1_s_at | D | 0.999999 | U95001UTR#1 RNU95001 Rattus norvegicus |
| | | | developmentally-regulated cardiac factor (DRCF-5) mRNA |
| X07686cds_s_at | D | 0.999997 | X07686cds RNLB6 Rat L1Rn B6 repetitive DNA element |
| X14254cds_g_at | D | 0.999994 | X14254cds RNMHC2I Rat mRNA for MHC class II- |
| | | | associated invariant chain |
| X15216cds_s_at | D | 0.999982 | X15216cds RRRPL21 R. rattus mRNA for ribosomal |
| | | | protein L21 |
| X17012mRNA_s_at | D | 0.999999 | X17012mRNA RNIGF2 Rat IGFII gene for insulin-like |
| | | | growth factor II |
| X51706cds_at | D | 0.998336 | X51706cds RRRPL9 Rat mRNA for ribosomal protein L9 |
| X52733cds_s_at | D | 0.999999 | X52733cds RRRPL27A Rat mRNA for ribosomal protein |
| | | | L27a |
| X56325mRNA_s_at | D | 1 | X56325mRNA RN2A1GL R. norvegicus 2-alpha-1 globin |
| | | | gene |
| X57529cds_s_at | D | 0.999885 | X57529cds RRRPS18A R. rattus ribosomal protein S18 |
| | | | mRNA |
| X58200mRNA_at | D | 0.999885 | X58200mRNA RRRPL23 Rat mRNA for ribosomal protein |
| | | | L23 |
| X58200mRNA_g_at | D | 0.999996 | X58200mRNA RRRPL23 Rat mRNA for ribosomal protein |
| | | | L23 |
| X58389cds_s_at | D | 0.999667 | X58389cds RRRPL17 Rat mRNA for ribosomal protein |
| | | | L17 |
| X61381cds_s_at | D | 0.999835 | X61381cds RRIIMRNA R. rattus interferon induced |
| | | | mRNA |
| X62146cds_g_at | D | 0.998124 | X62146cds RRRPL11 R. rattus mRNA for ribosomal |
| | | | protein L11 |
| X62660mRNA_g_at | D | 0.999996 | X62660mRNA RRGTS8 R. rattus mRNA for glutathione |
| | | | transferase subunit 8 |
| X77934cds_at | D | 0.999998 | X77934cds RNWAPLP2 R. norvegicus (Wistar) mRNA for |
| | | | amyloid precursor-like protein 2 |
| X77934cds_g_at | D | 0.999908 | X77934cds RNWAPLP2 R. norvegicus (Wistar) mRNA for |
| | | | amyloid precursor-like protein 2 |
| E03082cds_s_at | D | 0.999747 | E03082cds gDNA encoding rat nerve growth factor |
| M92075_at | D | 0.999998 | M92075 Rat meotropic glutamate receptor 2 mRNA |
| M96375_s_at | D | 0.999996 | M96375 Rattus norvegicus non-processed neurexin I-beta |
| | | | mRNA |
| M33025_s_at | D | 0.999592 | M33025 Rattus norvegicus parathymosin mRNA |
| J04488_at | D | 1 | J04488 Rat prostaglandin D synthetase mRNA |
| M86514_at | D | 0.99971 | M86514 Rat proline-rich protein mRNA |
| M60666_s_at | D | 0.999747 | M60666 Rat alpha-tropomyosin 2 mRNA |
| U07619_at | D | 0.999159 | U07619 Rattus norvegicus Sprague-Dawley tissue factor |
| | | | protein mRNA |
| U17919_s_at | D | 0.999941 | U17919 Rattus norvegicus allograft inflammatory factor-1 |
| | | | mRNA |
| U17607_at | D | 0.999957 | U17607 Rattus norvegicus CCAAT binding transcription |
| | | | factor CBF subunit C mRNA |
| X78593_at | D | 0.999991 | X78593 R. norvegicus mRNA for glycerol-3-phosphate |
| | | | dehydrogenase |
| U92564_at | D | 0.999977 | U92564 Rattus norvegicus Olf-1/EBF associated Zn finger |
| | | | protein Roaz mRNA |
| U92564_g_at | D | 0.999937 | U92564 Rattus norvegicus Olf-1/EBF associated Zn finger |
| | | | protein Roaz mRNA |
| U95178_s_at | D | 0.999991 | U95178 Rattus norvegicus DOC-2 p59 isoform mRNA |
| M15882_g_at | D | 0.999987 | M15882 Rat clathrin light chain (LCA1) mRNA |
| D64046_at | D | 0.997625 | D64046 Rat mRNA for phosphatidylinositol 3-kinase p85 |
| | | | beta subunit |
| rc_AI639098_at | D | 0.999893 | |
| rc_AI639109_at | D | 0.99999 | |
| rc_AI639111_at | D | 0.999893 | |
| rc_AI639119_at | D | 0.999667 | |
| rc_AI639155_at | D | 1 | |
| rc_AI639203_at | D | 0.999159 | |
| rc_AI639208_at | D | 0.999563 | |
| rc_AI639338_at | D | 0.999941 | |
| rc_AI639339_at | D | 0.99999 | |
| rc_AI639374_at | D | 0.998696 | |
| rc_AI639411_at | D | 0.998009 | |
| rc_AI639411_g_at | D | 0.999104 | |
| rc_AI639534_at | D | 0.999045 | |
| rc_AA859896_at | D | 0.999795 | |
| rc_AA892775_at | D | 0.999999 | |
| rc_AA799778_at | D | 0.999997 | |
| rc_AA851749_s_at | D | 0.997625 | |
| rc_AA852004_s_at | D | 0.997625 | |
| rc_AA900769_s_at | D | 0.999969 | |
| rc_AA900900_s_at | D | 0.999045 | |
| rc_AA944007_at | D | 0.998434 | |
| rc_AA946384_at | D | 0.999994 | |
| rc_AA955388_s_at | D | 0.999747 | |
| rc_AA965264_at | D | 0.99996 | |
| rc_AA998983_at | D | 0.999975 | |
| rc_AI007824_at | D | 0.999501 | |
| rc_AI008641_at | D | 0.999993 | |
| rc_AI012030_at | D | 0.999993 | |
| rc_AI012275_at | D | 0.999795 | |
| rc_AA817997_g_at | D | 0.999467 | |
| rc_AI103074_at | D | 0.999989 | |
| rc_AI103236_at | D | 0.999946 | |
| rc_AI104544_at | D | 0.99943 | |
| rc_AI172064_at | D | 0.999969 | |
| rc_AI175776_s_at | D | 0.999997 | |
| rc_AI176461_s_at | D | 0.999045 | |
| rc_AI178207_at | D | 0.999963 | |
| rc_AI179576_s_at | D | 0.999999 | |
| rc_AI231292_at | D | 0.999921 | |
| rc_AI232374_g_at | D | 0.999563 | |
| rc_AI232379_at | D | 0.999992 | |
| rc_AI233749_at | D | 0.999994 | |
| rc_AI234146_at | D | 0.999982 | |
| rc_AA817854_s_at | D | 0.999998 | |
| M36074_at | D | 0.998124 | M36074 Rat mineralocorticoid receptor (MR) mRNA |
| M11071_f_at | D | 0.999211 | M11071 Rat MHC class I cell surface antigen mRNA |
| M15562_at | D | 0.999932 | M15562 Rat MHC class II RT1.u-D-alpha chain mRNA |
| M15562_g_at | D | 0.999993 | M15562 Rat MHC class II RT1.u-D-alpha chain mRNA |
| M34043_at | D | 0.999921 | M34043 Rat thymosin beta-4 mRNA |
| M17419_at | D | 0.999351 | M17419 Rat ribosomal protein L5 mRNA |
| M84716_at | D | 0.999998 | M84716 R. norvegicus putative v-fos transformation |
| | | | effector protein (Fte-1) mRNA |
| U11760_at | D | 0.999954 | U11760 Rattus norvegicus transitional endoplasmic |
| | | | reticulum ATPase mRNA |
| U20796_at | D | 0.999954 | U20796 Rattus norvegicus nuclear receptor Rev-ErbA-beta |
| | | | mRNA |
| U09256_at | D | 0.999987 | U09256 Rattus norvegicus Sprague-Dawley transketolase |
| | | | mRNA |
| L26268_at | D | 0.999822 | L26268 Rattus norvegicus anti-proliferative factor (BTG1) |
| | | | mRNA |
| U25281_at | D | 0.998918 | U25281 Rattus norvegicus SH3 domain binding protein |
| | | | (CR16) mRNA |
| U48828_g_at | D | 0.999999 | U48828 Rattus norvegicus retroviral-like ovarian specific |
| | | | transcript 30-1 mRNA |
| X62322_g_at | D | 0.999996 | X62322 R. norvegicus mRNA for epithelin 1 and 2 |
| X73371_at | D | 0.999846 | X73371 R. norvegicus mRNA for Fc gamma receptor |
| X05834_at | D | 0.999747 | X05834 Rat fibronectin gene |
| X13016_at | D | 0.999999 | X13016 Rat mRNA for MRC OX-45 surface antigen |
| X52477_at | D | 0.999991 | X52477 Rat pcRC201 mRNA for pre-pro-complement C3 |
| AA944073_at | D | 0.99776 | |
| X13044_at | D | 0.999999 | X13044 Rat mRNA for MHC-associated invariant chain |
| | | | gamma |
| X13044_g_at | D | 1 | X13044 Rat mRNA for MHC-associated invariant chain |
| | | | gamma |
| U44845_at | D | 0.999822 | U44845 Rattus norvegicus vitronectin mRNA, |
| U60063_at | D | 0.998848 | U60063 Rattus norvegicus aldehyde dehydrogenase |
| | | | mRNA |
| D50436_at | D | 0.999159 | D50436 Rat mRNA for adrenodoxin |
| AF054586_at | D | 0.99943 | AF054586 Rattus norvegicus brain finger protein (BFP) |
| | | | mRNA |
| D84418_s_at | D | 0.999876 | D84418 Rat mRNA for chromosomal protein HMG2 |
| J04486_at | D | 0.999261 | J04486 Rat insulin growth factor-binding protein mRNA |
| L07281_at | D | 0.999729 | L07281 Rattus norvegicus carboxypeptidase E (CPE) gene |
| M19257_at | D | 0.998336 | M19257 Rat cytosolic retinol-binding protein (CRBP) |
| | | | mRNA |
| U88036_at | D | 0.999996 | U88036 Rattus norvegicus brain digoxin carrier protein |
| | | | mRNA |
| AF022083_s_at | D | 0.999997 | AF022083 Rattus norvegicus guanine nucleotide binding |
| | | | protein beta 1 subunit mRNA |
| J03179_at | D | 0.999988 | J03179 Rat D-binding protein mRNA |
| J03179_g_at | D | 0.999992 | J03179 Rat D-binding protein mRNA |
| L08505_at | D | 0.999846 | L08505 Rattus norvegicus cytoplasmic dynein heavy chain |
| | | | (MAP 1C), mRNA |
| L14851_at | D | 1 | L14851 Rattus norvegicus neurexin III-alpha gene |
| M91652complete_seq_g_at | D | 0.999307 | M91652completeSeq Rat glutamine synthetase (glnA) |
| | | | mRNA |
| rc_AA859805_at | D | 0.999729 | |
| rc_AA859902_at | D | 0.999941 | |
| rc_AA859919_at | D | 0.999941 | |
| rc_AA859919_g_at | D | 0.998696 | |
| rc_AA859994_at | D | 0.99776 | |
| rc_AA860043_at | D | 0.999857 | |
| rc_AA874943_at | D | 0.999563 | |
| rc_AA875059_at | D | 0.999957 | |
| rc_AA875102_at | D | 0.998336 | |
| rc_AA875531_s_at | D | 0.99978 | |
| rc_AA875577_at | D | 0.999533 | |
| rc_AA875633_at | D | 0.998233 | |
| rc_H31550_at | D | 0.99995 | |
| rc_H31990_at | D | 0.999957 | |
| rc_AA684631_at | D | 0.998009 | |
| rc_AA891054_at | D | 0.999999 | |
| rc_AA891107_at | D | 0.999846 | |
| rc_AA891725_at | D | 0.99996 | |
| rc_AA891802_at | D | 0.999846 | |
| rc_AA891872_at | D | 0.999563 | |
| rc_AA891922_at | D | 0.997625 | |
| rc_AA892137_at | D | 0.999966 | |
| rc_AA892376_at | D | 0.999954 | |
| rc_AA892470_at | D | 0.999996 | |
| rc_AA892557_g_at | D | 0.999211 | |
| rc_AA892570_at | D | 0.999392 | |
| rc_AA892616_at | D | 0.999045 | |
| rc_AA892675_at | D | 0.999999 | |
| rc_AA892854_at | D | 0.999999 | |
| rc_AA893002_at | D | 0.999351 | |
| rc_AA893351_at | D | 0.99776 | |
| rc_AA893357_at | D | 0.999392 | |
| rc_AA893699_s_at | D | 0.999747 | |
| rc_AA893743_at | D | 0.998233 | |
| rc_AA893939_g_at | D | 0.999999 | |
| rc_AA894207_g_at | D | 0.999997 | |
| rc_AA799511_g_at | D | 0.99776 | |
| rc_AA799594_at | D | 0.99943 | |
| rc_AA799755_at | D | 0.999981 | |
| rc_AA800029_at | D | 0.999846 | |
| rc_AA800126_at | D | 0.999999 | |
| rc_AA800318_at | D | 0.999618 | |
| rc_AA800622_at | D | 0.999747 | |
| rc_AA817964_s_at | I | 0.000955 | |
| rc_AI169372_at | I | 0.001082 | |
| rc_AI014091_at | I | 0.000841 | |
| rc_AI236601_at | I | 0.000001 | |
| rc_AA893711_at | I | 0.000177 | |
| rc_AA998683_g_at | I | 0.001017 | |
| rc_AA944014_at | I | 0.00004 | |
| rc_AA945867_at | I | 0 | |
| rc_AI175959_at | I | 0 | |
| rc_AI171166_at | I | 0.000034 | |
| D16308_at | I | 0.000467 | D16308 RATCLND2 Rat mRNA for cyclin D2 |
| D21799_at | I | 0.000693 | D21799 RATPSRC7I Rat mRNA for proteasome subunit |
| | | | RC7-Is |
| D21799_g_at | I | 0.000608 | D21799 RATPSRC7I Rat mRNA for proteasome subunit |
| | | | RC7-I |
| E01415cds_s_at | I | 0.000007 | E01415cds cDNA encoding rat glutathione S transferase |
| J02722cds_at | I | 0.000107 | J02722cds RATHOXA Rat heme oxygenase gene, |
| | | | complete cds |
| L16764_s_at | I | 0.002112 | L16764 RATHSP70A Rattus norvegicus heat shock protein |
| | | | 70 (HSP70) mRNA |
| L25925_s_at | I | 0 | L25925 RATCYLO2X Rat cyclooxygenase-2 mRNA |
| M11794cds#2_f_at | I | 0.000165 | M11794cds#2 RATMT12C Rat metallothionein-2 and |
| | | | metallothionein-1 genes |
| U03470_at | I | 0.000533 | U03470 Rattus norvegicus Fas antigen ligand mRNA |
| U30186_at | I | 0.000001 | U30186 Rattus norvegicus growth arrest and DNA- |
| | | | damage-inducible protein GADD153 mRNA |
| U38376_s_at | I | 0.001876 | U38376 Rattus norvegicus cytosolic phospholipase A2 |
| | | | mRNA |
| M14050_s_at | I | 0.001876 | M14050 Rat immunoglobulin heavy chain binding protein |
| | | | (BiP) mRNA |
| M55534mRNA_s_at | I | 0 | M55534mRNA Rat alpha-crystallin B chain mRNA |
| AF013144_at | I | 0.000027 | AF013144 Rattus norvegicus MAP-kinase phosphatase |
| | | | (cpg21) mRNA |
| U18982_s_at | I | 0.000841 | U18982 RNU18982 Rattus norvegicus fos-related antigen 2 |
| | | | (fra-2) mRNA |
| U20643mRNA#2_f_at | I | 0.000349 | U20643mRNA#2 RNU20643 Rattus norvegicus aldolase |
| | | | A gene |
| X17163cds_s_at | I | 0.000009 | X17163cds RSJUNAP1 Rat c-jun oncogene mRNA for |
| | | | transcription factor AP-1 |
| X82021cds_at | I | 0.000099 | X82021cds RNHSRP R. norvegicus mRNA for heat shock |
| | | | related protein |
| X94185cds_s_at | I | 0.000034 | X94185cds RNMKP3 R. norvegicus mRNA for dual |
| | | | specificity phosphatase, MKP-3 |
| AA108277_at | I | 0.000177 | |
| AA848218_at | I | 0.000043 | |
| AB000717exons#1- | I | 0 | AB000717exons#1-8 AB000716S2 Rattus rattus gene for |
| 8_{—s}_at | | | non-hepatic-type S-adenosylmethionine synthetase |
| AB001982_at | I | 0.001664 | AB001982 Rattus norvegicus mRNA for growth hormone |
| | | | secretagogue receptor type 1a |
| AB001982_g_at | I | 0.000079 | AB001982 Rattus norvegicus mRNA for growth hormone |
| | | | secretagogue receptor type 1a |
| AB005540_g_at | I | 0.000018 | AB005540 Rattus rattus mRNA for PCTAIRE2 |
| AB013130_at | I | 0.000006 | AB013130 Rattus norvegicus mRNA for synaptopodin |
| AB017140_g_at | I | 0.000955 | AB017140 Rattus norvegicus mRNA for PSD-Zip45 |
| AB018049_s_at | I | 0.001991 | AB018049 Rattus norvegicus mRNA for GM3 synthase |
| AB020504_at | I | 0.000092 | AB020504 Rattus norvegicus mRNA for PMF31 |
| AB020504_g_at | I | 0.001082 | AB020504 Rattus norvegicus mRNA for PMF31 |
| AB022209_s_at | I | 0.000608 | AB022209 Rattus norvegicus mRNA for ribonucleoprotein F |
| AF000899_g_at | I | 0.000007 | AF000899 RNP58S02 Rattus norvegicus p58/p45 mRNA |
| AF000900_g_at | I | 0.000271 | AF000900 Rattus norvegicus nucleoporin p45 (p58/p45) |
| | | | mRNA |
| AF000901_s_at | I | 0.001226 | AF000901 Rattus norvegicus p23 (p58/p45) mRNA |
| AF016387_g_at | I | 0.001566 | AF016387 Rattus norvegicus retinoid X receptor gamma |
| | | | (RXRgamma) mRNA |
| AF020045_at | I | 0.001991 | AF020045 Rattus norvegicus integrin alpha E1 mRNA |
| AF022819_at | I | 0.000003 | AF022819 Rattus norvegicus putative potassium channel |
| | | | TWIK mRNA |
| AF023087_s_at | I | 0.001566 | AF023087 Rattus norvegicus nerve growth factor induced |
| | | | factor A mRNA |
| AF030087UTR#1_g_{—} | I | 0.001474 | AF030087UTR#1 Rattus norvegicus activity and |
| at | | | neurotransmitter-induced early gene 2 (ania-2) mRNA |
| AF030088UTR#1_at | I | 0.000018 | AF030088UTR#1 Rattus norvegicus activity and |
| | | | neurotransmitter-induced early gene 3 (ania-3) mRNA |
| AF030089UTR#1_at | I | 0 | AF030089UTR#1 Rattus norvegicus activity and |
| | | | neurotransmitter-induced early gene 4 (ania-4) mRNA |
| AF034900mRNA_i_at | I | 0.000009 | AF034900mRNA Rattus norvegicus olfactory receptor-like |
| | | | protein (SCR D-7) gene |
| AF062740_at | I | 0 | AF062740 Rattus norvegicus pyruvate dehydrogenase |
| | | | phosphatase isoenzyme 1 mRNA |
| AF062740_g_at | I | 0 | AF062740 Rattus norvegicus pyruvate dehydrogenase |
| | | | phosphatase isoenzyme 1 mRNA |
| AF069782_at | I | 0.000001 | AF069782 Rattus sp. unknown mRNA |
| AF083418_at | I | 0.000955 | AF083418 Rattus norvegicus insulin receptor substrate-2 |
| | | | (IRS-2) mRNA |
| AF086624_s_at | I | 0.000205 | AF086624 Rattus norvegicus serine threonine kinase (pim- |
| | | | 3) mRNA, |
| AF087674_at | I | 0.000955 | AF087674 Rattus norvegicus insulin receptor substrate 2 |
| | | | (IRS-2) mRNA |
| AF091563_i_at | I | 0.00039 | AF091563 Rattus norvegicus isolate QIL-LD1 olfactory |
| | | | receptor mRNA |
| AF091570_s_at | I | 0.00022 | AF091570 Rattus norvegicus isolate HGL-SP2 olfactory |
| | | | receptor pseudogene, |
| AJ001290cds_at | I | 0.000004 | AJ001290cds RNSMIT Rattus norvegicus mRNA for |
| | | | sodium myo-inositol transporter (SMIT) |
| AJ004858_at | I | 0.000004 | AJ004858 RNAJ4858 Rattus norvegicus mRNA for Sry- |
| | | | related HMG-box protein Sox11 |
| AJ006855_s_at | I | 0.000533 | AJ006855 RNAJ6855 Rattus norvegicus mRNA for |
| | | | synaptojanin |
| AJ007016_at | I | 0.001767 | AJ007016 RNO7016 Rattus norvegicus mRNA for protein |
| | | | tyrosine phosphatase |
| D10938exon_s_at | I | 0.000001 | D10938exon RATSDBDNF Rat gene for brain-derived |
| | | | neurotrophic factor (BDNF) |
| D12769_at | I | 0.000004 | D12769 RATBTEB Rattus norvegicus mRNA for BTE |
| | | | binding protein |
| D12769_g_at | I | 0.000099 | D12769 RATBTEB Rattus norvegicus mRNA for BTE |
| | | | binding protein |
| D13417_at | I | 0.00001 | D13417 RATHES1A Rat mRNA for transcription factor |
| | | | HES-1 |
| D13417_g_at | I | 0.000015 | D13417 RATHES1A Rat mRNA for transcription factor |
| | | | HES-1 |
| D13962_g_at | I | 0.000008 | D13962 RATGLUT3 Rat mRNA for neuron glucose |
| | | | transporter |
| D45254_at | I | 0.000124 | D45254 RATCNABP Rat mRNA for cellular nucleic acid |
| | | | binding protein (CNBP) |
| D84477_at | I | 0.000649 | D84477 Rattus norvegicus mRNA for RhoA |
| L07380_at | I | 0.00224 | L07380 RATGHRFRG Rattus rattus (clone pGR2) growth |
| | | | hormone-releasing factor receptor mRNA sequence |
| L07925_g_at | I | 0.001991 | L07925 RATGNDSA Rattus rattus guanine nucleotide |
| | | | dissociation stimulator for a ras-related GTPase mRNA |
| L21995_s_at | I | 0.000789 | L21995 RATMOGX Rat myelin/oligodendrocyte |
| | | | glycoprotein (MOG) mRNA |
| L81137exon_s_at | I | 0.001474 | L81137exon RATRPS2R2A Rattus norvegicus (strain |
| | | | R21) Rps2r2 gene |
| L81138exon_i_at | I | 0.000432 | L81138exon RATRPS2R3A Rattus norvegicus (strain |
| | | | R21) Rps2r gene |
| M10088cds_s_at | I | 0.000382 | M10088cds RATENKB Rat prodynorphin |
| | | | (Preproenkephalin B) gene |
| M19648cds_f_at | I | 0.000608 | M19648cds RATKALB02 Rat renal kallikrein gene |
| M25638_s_at | I | 0.000037 | M25638 RATNFL Rat smallest neurofilament protein (NF- |
| | | | L) mRNA |
| M32783cds_i_at | I | 0.001892 | M32783cds RATENKA3 Rat dynorphin gene |
| M64377_f_at | I | 0.001017 | M64377 RATOLFPROC Rat olfactory protein mRNA |
| M74898cds_s_at | I | 0.000649 | M74898cds RATKCH Rat potassium channel protein |
| | | | mRNA |
| M92433exon#1- | I | 0 | M92433exon#1-2 RATNGFIC3E Rattus rattus NGFI-C |
| 2_{—s}_at | | | gene |
| S49491_s_at | I | 0 | S49491 proenkephalin [rats, heart, mRNA |
| S55933_i_at | I | 0.00004 | S55933 GABAA receptor alpha 4 subunit [rats, mRNA |
| S58644_at | I | 0.000003 | S58644 integrin beta 5 subunit mRNA |
| S60953_s_at | I | 0.001566 | S60953 trkC(ki39) = receptor tyrosine kinase |
| S63521_i_at | I | 0.000219 | S63521 glucose-regulated protein GRP78 |
| S63521_r_at | I | 0.000004 | S63521 glucose-regulated protein GRP78 |
| S65091_g_at | I | 0.000003 | S65091 cyclic AMP-regulated phosphoprotein |
| S66024_at | I | 0.001082 | S66024 transcriptional repressor CREM |
| S66024_g_at | I | 0.001474 | S66024 transcriptional repressor CREM |
| S67722_s_at | I | 0 | S67722 cyclooxygenase isoform COX-2 |
| S71196mRNA_s_at | I | 0.000004 | S71196mRNA S1 brain-derived neurotrophic factor |
| S76758_i_at | I | 0 | S76758 BDNF = brain-derived neurotrophic factor |
| S77528cds_s_at | I | 0.000004 | S77528cds rNFIL-6 = C/EBP-related transcription factor |
| S80345_s_at | I | 0.00029 | S80345 VHL = von Hippel-Lindau tumor suppressor gene |
| S82649_r_at | I | 0 | S82649 Narp = neuronal activity-regulated pentraxin |
| S82649_s_at | I | 0 | S82649 Narp = neuronal activity-regulated pentraxin |
| U02320_s_at | I | 0.000499 | U02320 RNU02320 Rattus norvegicus clone ndf40 neu |
| | | | differentiation factor mRNA |
| U02506UTR#1_s_at | I | 0.000649 | U02506UTR#1 RNU02506 Rattus norvegicus clone 7 |
| | | | polymeric immunoglobulin receptor mRNA |
| U12428_at | I | 0.000001 | U12428 RNU12428 Rattus norvegicus Sprague-Dawley |
| | | | zinc-finger transcription factor Egr3/Pilot mRNA |
| U75397UTR#1_s_at | I | 0.000115 | U75397UTR#1 RNKROX2 Rattus norvegicus Krox-24 |
| | | | mRNA |
| U75411cds_g_at | I | 0.00224 | U75411cds RNU75411 Rattus norvegicus anti-idiotype |
| | | | immunoglobulin M light chain gene |
| U75921UTR#1_at | I | 0.000311 | U75921UTR#1 RNAPCBP3 Rattus norvegicus APC |
| | | | binding protein EB1 mRNA |
| U75927UTR#1_at | I | 0.001991 | U75927UTR#1 RNCOVII2 Rattus norvegicus cytochrome |
| | | | oxidase subunit VIIa mRNA |
| U77931_at | I | 0.000408 | |
| U88324_at | I | 0.000133 | U88324 RNU88324 Rattus norvegicus G protein beta1 |
| | | | subunit (rGb1) mRNA |
| U91561_g_at | I | 0.000311 | U91561 RNU91561 Rattus norvegicus pyridoxine 5- |
| | | | phosphate oxidase mRNA |
| V01201cds#3_at | I | 0.001152 | V01201cds#3 RESSV1 Simian sarcoma virus proviral |
| | | | sequence with genes coding for envelope protein (env) and |
| | | | polymerase (pol) |
| X02322Poly_A_Site# | I | 0.001226 | X02322Poly_Aite#1 RNGHRF5 Rat gene for growth |
| 1_s_at | | | hormone-releasing factor |
| X07266cds_s_at | I | 0.001566 | X07266cds RRG33A Rat mRNA for gene 33 polypeptide |
| X59677mRNA_s_at | I | 0.000143 | X59677mRNA RSM2798 Rattus sp. cDNA for M2 gene |
| X60351cds_s_at | I | 0.000107 | X60351cds RRLENSABC R. rattus mRNA for alpha B- |
| | | | crystallin (ocular lens tissue) |
| X89694cds_at | I | 0.00224 | X89694cds RNTPCR04P R. norvegicus mRNA for TPCR04 |
| | | | protein |
| Y14933mRNA_s_at | I | 0.001566 | Y14933mRNA RNHNF6B R. norvegicus mRNA for |
| | | | hepatocyte nuclear factor 6 beta |
| Z15123exon#5_s_at | I | 0 | Z15123exon#5 RNAMDX48 R. norvegicus S- |
| | | | adenosylmethionine decarboxylase gene |
| Z21935cds_at | I | 0.000896 | Z21935cds RNPROKINA R. norvegicus protein kinase |
| | | | rMNK2 |
| E12625cds_at | I | 0.000236 | E12625cds cDNA encoding a rat novel protein which is |
| | | | expressed with nerve injury |
| M92430_at | I | 0.00224 | M92430 Rat neu differentiation factor mRNA |
| J05571_s_at | I | 0.000031 | J05571 Rat S-adenosylmethionine synthetase mRNA |
| M74223_at | I | 0.000002 | M74223 Rat VGF mRNA |
| M35535_at | I | 0.001017 | M35535 R. norvegicus gastric (H+, K+)-ATPase beta- |
| | | | subunit mRNA |
| U19866_at | I | 0 | U19866 Rattus norvegicus growth factor (Arc) mRNA |
| U24174_at | I | 0.000054 | U24174 Rattus norvegicus p21 (WAF1) mRNA |
| U24282_g_at | I | 0.001387 | U24282 Rattus norvegicus type III iodothyronine |
| | | | deiodinase (dioIII) mRNA |
| L19180_at | I | 0.001152 | L19180 Rat receptor-linked protein tyrosine phosphatase |
| | | | (PTP-P1) mRNA, |
| U24441_at | I | 0.000124 | U24441 Rattus norvegicus gelatinase B (GelB) mRNA |
| U02315_at | I | 0.001566 | U02315 Rattus norvegicus clone ndf04 neu differentiation |
| | | | factor mRNA |
| X78949_at | I | 0.00022 | X78949 R. norvegicus mRNA for prolyl 4-hydroxylase |
| | | | alpha subunit |
| X15580complete_seq_{—} | I | 0.000739 | X15580completeSeq R. norvegicus mRNA for 6- |
| s_at | | | phosphofructo-2-kinase/fructose-2,6-bisphosphatase |
| X53231_at | I | 0.000205 | X53231 Rat mRNA for preoptic regulatory factor-1 |
| | | | (PORF-1) |
| X87635_at | I | 0.001304 | X87635 R. norvegicus mRNA for inwardly rectifying |
| | | | potassium channel |
| U33471_at | I | 0.00057 | U33471 Rattus norvegicus balloon angioplasty responsive |
| | | | transcript (BART-1) mRNA |
| U78102_at | I | 0.000079 | U78102 Rattus norvegicus krox20 mRNA |
| L19341_at | I | 0.000177 | L19341 Rattus norvegicus activin type I receptor mRNA |
| D38072_at | I | 0.001082 | D38072 Rat mRNA for protein tyrosine phosphatase |
| AF052540_s_at | I | 0.001387 | AF052540 Rattus norvegicus calpain isoform Lp85 mRNA |
| AJ001380_at | I | 0.000107 | AJ001380 Rattus norvegicus Tspy pseudogene |
| Z69594_at | I | 0.00224 | Z69594 R. norvegicus platelet glycoprotein V gene |
| D14447_at | I | 0.002375 | D14447 Rat Max mRNA |
| AF009330_at | I | 0.000034 | AF009330 Rattus norvegicus enhancer-of-split and hairy- |
| | | | related protein 2 (SHARP-2) mRNA |
| AB003726_at | I | 0 | AB003726 Rattus norvegicus mRNA for Vesl |
| AF026529_s_at | I | 0.000382 | AF026529 Rattus norvegicus stathmin-like-protein splice |
| | | | variant RB3 mRNA |
| AF036335_at | I | 0.000019 | AF036335 Rattus norvegicus NonO/p54nrb homolog |
| | | | mRNA |
| AF036335_g_at | I | 0.000001 | AF036335 Rattus norvegicus NonO/p54nrb homolog |
| | | | mRNA |
| M75281_at | I | 0.001876 | M75281 Rat cystatin S (CysS) gene |
| M84361_at | I | 0.000311 | M84361 Rat CSF-1 protein mRNA |
| rc_AI638966_r_at | I | 0.000376 | |
| rc_AI639028_at | I | 0.00224 | |
| rc_AI639039_at | I | 0.00224 | |
| rc_AI639058_s_at | I | 0 | |
| rc_AI639257_at | I | 0.000001 | |
| rc_AI639323_at | I | 0.001304 | |
| rc_AI639325_at | I | 0.001226 | |
| rc_AI639413_at | I | 0.001566 | |
| rc_AI639434_at | I | 0.00224 | |
| rc_AI639468_at | I | 0.000789 | |
| rc_AI639472_i_at | I | 0.000154 | |
| rc_AA875582_at | I | 0.001991 | |
| rc_AA891527_at | I | 0.000124 | |
| rc_AA924925_at | I | 0.00001 | |
| rc_AA965147_at | I | 0.00004 | |
| rc_AA965154_at | I | 0.000154 | |
| rc_AA997865_at | I | 0.002375 | |
| rc_AI008131_s_at | I | 0 | |
| rc_AI009605_at | I | 0.000003 | |
| rc_AI014135_at | I | 0.00005 | |
| rc_AI014163_at | I | 0 | |
| rc_AI030286_s_at | I | 0 | |
| rc_AI043631_s_at | I | 0.000074 | |
| rc_AI044508_s_at | I | 0.000003 | |
| rc_AI044739_at | I | 0.001767 | |
| rc_AI071299_at | I | 0 | |
| rc_AI071498_at | I | 0.00057 | |
| rc_AI113289_s_at | I | 0.000205 | |
| rc_AI105198_i_at | I | 0.001544 | |
| rc_AI169327_at | I | 0.000007 | |
| rc_AI169327_g_at | I | 0 | |
| rc_AI102562_at | I | 0.000014 | |
| rc_AI170776_s_at | I | 0.000001 | |
| rc_AI172476_at | I | 0 | |
| rc_AA818982_g_at | I | 0.000271 | |
| rc_AI176456_at | I | 0 | |
| rc_AI176662_s_at | I | 0.001152 | |
| rc_AI176710_at | I | 0 | |
| rc_AI177404_at | I | 0.001017 | |
| rc_AI177503_at | I | 0.000789 | |
| rc_AI179610_at | I | 0.000019 | |
| rc_AI229031_s_at | I | 0.000191 | |
| rc_AI231375_at | I | 0.000608 | |
| rc_AI232284_i_at | I | 0.001427 | |
| rc_AI235890_s_at | I | 0 | |
| M58587_at | I | 0.000009 | M58587 Rat liver interleukin 6 receptor ligand binding |
| | | | chain mRNA |
| M25584_at | I | 0.000054 | M25584 Rat insulin 1 gene |
| M29014_at | I | 0.001664 | M29014 Rat insulin receptor mRNA |
| M31603_at | I | 0.000001 | M31603 Rat parathyroid-like peptide mRNA |
| L19699_g_at | I | 0.000253 | L19699 Rat GTP-binding protein (ral B) mRNA |
| L25633_at | I | 0.000007 | L25633 Rattus norvegicus neuroendocrine-specific protein |
| | | | (RESP18) mRNA |
| L25633_g_at | I | 0.000003 | L25633 Rattus norvegicus neuroendocrine-specific protein |
| | | | (RESP18) mRNA |
| L01624_at | I | 0.000001 | L01624 Rattus norvegicus serum and glucocorticoid- |
| | | | regulated kinase (sgk) mRNA |
| M55291_at | I | 0.000001 | M55291 Rat neural receptor protein-tyrosine kinase (trkB) |
| | | | mRNA |
| M18416_at | I | 0.00029 | M18416 Rat nerve growth factor-induced (NGFI-A) gene |
| J04563_at | I | 0.000054 | J04563 Rat cAMP phosphodiesterase mRNA |
| J04792_at | I | 0.000408 | J04792 Rattus norvegicus ornithine decarboxylase (ODC) |
| | | | gene |
| U19614_g_at | I | 0.000437 | U19614 Rattus norvegicus lamina-associated polypeptide |
| | | | 1C (LAP1C) mRNA |
| U14746_at | I | 0.000009 | U14746 Rattus norvegicus VHL protein (VHL) mRNA |
| U49062_at | I | 0.001304 | U49062 Rattus norvegicus heat sle antigen CD24 mRNA |
| U49062_g_at | I | 0.000107 | U49062 Rattus norvegicus heat sle antigen CD24 mRNA |
| X83399_at | I | 0.00022 | |
| X67108_at | I | 0.000789 | X67108 R. norvegicus mRNA for brain-derived |
| | | | neurotrophic factor (exon IV) |
| X57281_at | I | 0.002375 | X57281 Rat NGlyR mRNA for neonatal glycine receptor |
| X86086_g_at | I | 0.000115 | X86086 R. norvegicus RNA for annexin VI |
| X60769mRNA_at | I | 0.000001 | X60769mRNA Rat sfb mRNA for silencer factor B |
| X71898_at | I | 0.000027 | X71898 R. norvegicus uPAR-1 mRNA for urinary |
| | | | plasminogen activator receptor 1 |
| X95466_at | I | 0.000037 | X95466 R. norvegicus mRNA for CPG2 protein |
| U42627_at | I | 0.000177 | U42627 Rattus norvegicus dual-specificity protein tyrosine |
| | | | phosphatase (rVH6) mRNA |
| U63840_at | I | 0.001566 | U63840 Rattus norvegicus nucleoporin p54 mRNA, |
| U72620_at | I | 0.000015 | U72620 Rattus norvegicus Lot1 mRNA |
| U53859_at | I | 0.000034 | U53859 Rattus norvegicus calpain small subunit (css1) |
| | | | mRNA |
| D45920_at | I | 0.000004 | D45920 Rat mRNA for 130 kDa-Ins(1, 4, 5)P3 binding |
| | | | protein |
| U67140_at | I | 0.000018 | U67140 Rattus norvegicus PSD-95/SAP90-associated |
| | | | protein-4 mRNA |
| L12383_at | I | 0.000043 | L12383 Rattus norvegicus ADP-ribosylation factor 4 |
| | | | mRNA |
| U88958_at | I | 0.000001 | U88958 Rattus norvegicus neuritin mRNA, |
| AF029240_at | I | 0.000955 | AF029240 Rattus norvegicus MHC class Ib RT1.S3 |
| | | | (RT1.S3) gene |
| M57664_at | I | 0.000031 | M57664 Rat cretine kinase-B (CKB) mRNA |
| M57664_g_at | I | 0.000001 | M57664 Rat cretine kinase-B (CKB) mRNA |
| AF009329_at | I | 0.000001 | AF009329 Rattus norvegicus enhancer-of-split and hairy- |
| | | | related protein 1 (SHARP-1) mRNA |
| AF016296_at | I | 0 | AF016296 Rattus norvegicus neuropilin mRNA |
| AF019974_at | I | 0.000008 | AF019974 Rattus norvegicus chromogranin B (Chgb) |
| | | | mRNA, |
| AF029107_at | I | 0.000059 | AF029107 Rattus norvegicus Mint2 mRNA |
| M33648_g_at | I | 0.000043 | M33648 Rat mitochondrial 3-hydroxy-3-methylglutaryl- |
| | | | CoA synthase mRNA |
| rc_AA860030_s_at | I | 0.000031 | |
| rc_AA866299_g_at | I | 0.000382 | |
| rc_AA866452_s_at | I | 0.001566 | |
| rc_AA875025_at | I | 0.000205 | |
| rc_AA875032_at | I | 0 | |
| rc_AA875069_at | I | 0.000011 | |
| rc_AA875468_at | I | 0.000499 | |
| rc_H31287_at | I | 0.001876 | |
| rc_H31692_at | I | 0.002375 | |
| rc_AA891542_at | I | 0.000649 | |
| rc_AA891595_at | I | 0.000124 | |
| rc_AA891716_at | I | 0.001991 | |
| rc_AA891735_at | I | 0.001226 | |
| rc_AA892319_at | I | 0.000693 | |
| rc_AA892647_at | I | 0.000006 | |
| rc_AA892799_i_at | I | 0.001304 | |
| rc_AA892814_s_at | I | 0.000154 | |
| rc_AA892942_at | I | 0.000311 | |
| rc_AA893172_at | I | 0.001304 | |
| rc_AA893202_at | I | 0.00057 | |
| rc_AA893206_at | I | 0.002375 | |
| rc_AA893869_g_at | I | 0.001152 | |
| rc_AA893870_g_at | I | 0 | |
| rc_AA894148_s_at | I | 0.000236 | |
| rc_H33619_at | I | 0.00057 | |
| rc_AA799406_at | I | 0.000154 | |
| rc_AA799538_at | I | 0.001566 | |
| rc_AA800036_at | I | 0.000001 | |
| rc_AA800549_at | I | 0.000437 | |
| rc_AA800551_at | I | 0.000009 | |
| rc_AA800701_at | I | 0.000015 | |
| rc_AA800737_at | I | 0.001664 | |
| rc_AA800840_at | I | 0.000841 | |

The present invention is not limited to the embodiments described above, but is capable of variation and modification within the scope of the appended claims.

1. A composition of matter comprising a collection of two or more probes for detecting expression of two or more seizure-induced genes, wherein the probes comprise one or more of:
a) oligonucleotides or polynucleotides that specifically hybridize to two or more of SEQ ID NOS: 1-48; or
b) polypeptide binding agents that specifically bind to polypeptides produced by expression of two or more nucleic acid molecules comprising sequences selected from the group consisting of SEQ ID NOS: 1-48.

2. The composition of claim 1, comprising a collection of five or more probes for detecting expression of five or more seizure-induced genes.

3. The composition of claim 1, comprising a collection of ten or more probes for detecting expression of ten or more seizure-induced genes.

4. The composition of claim 1, wherein the probes are affixed to a solid support at known locations.

5. The composition of claim 1, wherein the polypeptide binding agents are antibodies.

6. A test kit for analyzing expression of seizure-induced genes, comprising a container containing a collection of two or more probes for detecting expression of two or more seizure-induced genes, wherein the probes comprise one or more of:
a) oligonucleotides or polynucleotides that specifically hybridize to two or more of SEQ ID NOS: 1-48; or
c) polypeptide binding agents that specifically bind to polypeptides produced by expression of two or more nucleic acid molecules comprising sequences selected from the group consisting of SEQ ID NOS: 1-48;
and instructions for performing a gene expression assay.

7. The test kit of claim 6, comprising a collection of five or more probes for detecting expression of five or more seizure-induced genes.

8. The test kit of claim 6, comprising a collection of ten or more probes for detecting expression of ten or more seizure-induced genes.

9. The test kit of claim 6, wherein the probes are affixed to a solid support at known locations.

10. The test kit of claim 6, wherein the polypeptide binding agents are antibodies.

11. A device for detecting expression of a plurality of seizure-induced genes, comprising a solid support to which is affixed an array comprising a plurality of probes specific for transcription or translation products of the seizure induced genes, wherein the probes comprise either:
a) a plurality of oligonucleotides or polynucleotides, each of which specifically hybridizes to a different sequence selected from the group consisting of SEQ ID NOS: 1-48, or
b) a plurality of polypeptide binding agents, each of which specifically binds to a different polypeptide or fragment thereof produced by expression of a nucleic acid molecule comprising a sequence selected from the group consisting of SEQ ID NOS: 1-48.

12. The device of claim 11, wherein the polypeptide binding agents are antibodies.

13. An isolated nucleic acid molecule comprising a seizure-induced gene, mRNA or cDNA produced from the seizure induced gene, wherein the nucleic acid molecule comprises a sequence selected from the group consisting of SEQ ID NOS: 29-48.

14. A polypeptide encoded by the nucleic acid molecule of claim 13.

15. A method for measuring the effect of a test compound on activity of a polypeptide produced by expression of a seizure induced gene, wherein the polypeptide is selected from the group consisting of: Bach1, Interferon Related Developmental Regulator (IFRD1/PC4), Cis-Golgi SNARE p28, TFIID subunit TAFII55, FGF-Inducible gene (FIN14) product, Esterase-D or S-formylglutathione hydrolase, -L-fucosidase, Integrin 6 subunit, Macrophilin (microphilin and actin filament crosslinker protein or ACF7), Synaptogyrin 3, Metallothionine I and II, and GM3 synthase (or Sialytransferase 9), the method comprising measuring a biological activity of the polypeptide in the presence or absence of the test compound, wherein a change in the biological activity in the presence of the test compound is indicative of an effect of the test compound on activity of the polypeptide.

16. A method for measuring the effect of a test compound on expression of a seizure-induced gene selected from the group consisting of Bach1, MafK, Interferon Related Developmental Regulator (IFRD1/PC4), Cis-Golgi SNARE p28, TFIID subunit TAFII55, FGF-Inducible gene (FIN14), Esterase-D or S-formylglutathione hydrolase, -L-fucosidase, Integrin 6 subunit, Macrophilin (microphilin and actin filament crosslinker protein) or ACF7, Synaptogyrin 3, Metallothionine I and II, and GM3 synthase or Sialytransferase 9, the method comprising measuring production of transcription or translation products produced by expression of the gene in the presence or absence of the test compound, wherein a change in the production of transcription or translation products in the presence of the test compound is indicative of an effect of the test compound on expression of the gene.

17. The method of claim 16, wherein the gene expression is measured by providing a DNA construct comprising a reporter gene coding sequence operably linked to transcription regulatory sequences of the seizure-induced gene, and measuring formation of a reporter gene product in the presence or absence of the test compound.

18. The method of claim 16, wherein the gene is located within a cultured cell and the cultured cell is contacted with the test compound.

19. The method of claim 18, wherein the cultured cell is a cell line derived from primary neuronal or glial cells.

20. The method of claim 16, wherein the gene is located within a mammalian subject and the test compound is administered to the subject.

21. A method for measuring the effect of a test compound on the expression profile of a plurality of seizure-induced genes comprising two or more of SEQ ID NOS: 1-48, the method comprising measuring production of transcription or translation products produced by expression of the plurality of genes in the presence or absence of the test compound, wherein a change in the production of transcription or translation products of any of the genes in the presence of the test compound is indicative of an effect of the test compound on the expression profile of the plurality of seizure-induced genes.

22. The method of claim 21, wherein the plurality of genes is located within a cultured cell and the cultured cell is contacted with the test compound.

23. The method of claim 22, wherein the cultured cell is a cell line derived from primary neuronal or glial cells.

24. The method of claim 23, wherein the cells are treated with kainic acid prior to, concurrently with, or after exposure to the test compound.

25. The method of claim 24, wherein the cells are further treated with an NMDA receptor antagonist prior to, concurrently with, or after exposure to the test compound.

26. The method of claim 21, wherein the gene is located within a mammalian subject and the test compound is administered to the subject.

27. The method of claim 26, wherein the mammalian subject is an animal and the animal is subjected to inducement of a seizure prior to, concurrently with, or after administration of the test compound.

28. The method of claim 27, wherein the animal is further treated with an NMDA receptor antagonist prior to, concurrently with, or after exposure to the test compound.

29. The method of claim 27, wherein the mammalian subject is an animal and the animal is bred or genetically modified to exhibit slow kindling as a result of repeated seizure inducement.

30. The method of claim 27, wherein the mammalian subject is an animal and the animal is bred or genetically modified to exhibit fast kindling as a result of repeated seizure inducement.

31. The method of claim 27, adapted to establish an NMDA receptor-dependent and an NMDA receptor-independent gene expression profile in the absence of the test compound, wherein the animal is subjected to inducement of a seizure with or without administration of an NMDA receptor antagonist, and production of transcription or translation products produced by expression of each of a plurality of seizure-induced genes having sequences comprising SEQ ID NOS: 1-48 is measured, and the differences in production of transcription or translation products of each of the genes in the presence versus absence of the NMDA inhibitor is observed, thereby generating the NMDA receptor-dependent and NMDA receptor-independent gene expression profile of the seizure-induced genes.

32. A method to diagnose or develop a prognosis for a subject who has had a seizure, the method comprising:
a) obtaining a sample of neural cells from the subject;
b) measuring production of transcription or translation products produced by expression of two or more of a plurality of seizure-induced genes having sequences comprising SEQ ID NOS: 1-48;
c) determining whether any of the transcription or translation products of the seizure-induced genes is elevated as compared with a known control, wherein an elevation in transcription or translation products of any of the seizure-induced genes is indicative of the subject having had a seizure.

33. The method of claim 32, further comprising assessing whether the increased seizure-induced gene expression in the subject is characterized as NMDA-dependent or NMDA-independent gene expression.

34. A method of determining if a test compound affects NMDA-dependent, but not NMDA-independent seizure-induced gene expression in an animal subject, comprising the steps of:
a) establishing an NMDA receptor-dependent and an NMDA receptor-independent gene expression profile in the absence of the test compound by:
i) inducing a seizure in one animal in the presence of an NMDA receptor antagonist and inducing a seizure in another animal in the absence of an NMDA receptor antagonist;
ii) obtaining samples of transcription or translation products from neural cells of each animal; and
iii) identifying transcription or translation products that are differentially produced in the neural cells of animals induced to seizure in the presence of the NMDA receptor antagonist, as compared to in the absence of the NMDA receptor antagonist, thereby establishing the NMDA receptor-dependent and NMDA-receptor independent gene expression profiles and identifying a population of NMDA receptor-associated seizure-induced genes;
b) repeating step a) in the presence of the test compound, wherein differences in expression of one or more of the NMDA receptor-associated seizure-induced genes in the presence of the test compound is indicative that the test compound affects NMDA receptor-dependent but not NMDA receptor-independent seizure-induced gene expression.

35. The method of claim 34, wherein the gene expression profiles are generated by interrogating the samples of transcription or translation products on an array that represents all expressed genes in the animal.

36. The method of claim 35, wherein the gene expression profiles are generated by interrogating the samples of transcription or translation products on an array that represents nucleic acids comprising SEQ ID NOS: 1-48 or polypeptides encoded by genes comprising SEQ ID NOS: 1-48.

37. The method of claim 34, wherein the animal is a rat.

38. The method of claim 34, wherein the NMDA receptor antagonist is MK801.


SEQUENCE LISTING (48 sequences)

SEQ ID NO: 1 - DNA, 100 bp, Rattus sp.
  misc_feature (33)..(33): n is a, c, g, or t
gccatggtaatcctgctcagtacgagaggaacngcagtctagactattggtgtatgtgcttggctgaggagccaatggggccacagtaccatctgtggat

SEQ ID NO: 2 - DNA, 99 bp, Rattus sp.
actgttttactctgcagcccaggaaagcccacatcgcctgactgctcgtatgtgtgtaggaccgacaggaatagagcccgtgagtccagcagaatcctg

SEQ ID NO: 3 - DNA, 100 bp, Rattus sp.
agcttggtgaacttagataatttttgatgtagggatgggttatattttaatttaatgtacagtgttacaaattaatgagttctttattcctgtaaaaata

SEQ ID NO: 4 - DNA, 102 bp, Rattus sp.
cgtaagtaggcagaatcaacccatgcttttattttcatggcatataaaaattttatatgaaggtattgatgtacagtgctatccccatggctgtaagagagg

SEQ ID NO: 5 - DNA, 100 bp, Rattus sp.
aagcttccagcaacaaaccaataacatactactcaacataatactcatttcactagccataaaatccggactagccccattccactactgactacccgaa

SEQ ID NO: 6 - DNA, 89 bp, Rattus sp.
agcagcagcagcgaagatgaagaagatgtaaacatgattgacactgaagaggatctggagagacagctacaggacaagctaaatgagtc

SEQ ID NO: 7 - DNA, 104 bp, Rattus sp.
ttggtgaacgtaaagattgaccctcaagtcactgtagctttagtacttgcttattgtattagtttagatgctagcaccgcatgtgctctgcatatttctggttt

SEQ ID NO: 8 - DNA, 149 bp, Rattus sp.
cgacctgtctcgtgaagtcctactcgggtccacagattgacatactaattgatcaggggaaagatgacggagttctctcgaatggccagttactcctgacaacttcagcatcgctgcctgcacagaaaagaaaatccctgttgtttttt

SEQ ID NO: 9 - DNA, 131 bp, Rattus sp.
ggaaaggaagctactttattcacgataatccttaagagacagcagcacactgggccctctatccttgagctccagcgatcagttcacttttgtcagctttagagtccaggcagttctccttccattcctag

SEQ ID NO: 10 - DNA, 144 bp, Rattus sp.
acgatgctaggaatggaaggagaactgcctggactctaaagctgacaaaagtgaactgatcgcaggagctcaaggatagagggcccagtgtgctgctgtctcttaaggattatcgtgaataaagtagcttcctttccaaaaaaa

SEQ ID NO: 11 - DNA, 144 bp, Rattus sp.
ggccttacgaatgttaacagtttcaactatacatgagaacgacacaacaacatttagtttgcttatgaacttcataaaagaacatgtttaatcttcattcttgaaatccatttcattgcattgtcaaatgataatttcagtgtg

SEQ ID NO: 12 - DNA, 94 bp, Rattus sp.
cggggttatcagtgtgtttctcgtcgttgatctctcgctcttctttccgaatccgcaccgcatggtatcggggaacactgtcatcgacctagag

SEQ ID NO: 13 - DNA, 87 bp, Rattus sp.
gaattcttattttttttaatataatttatcttgctgatatagcaagtcaagtttataaagaagatgcatttaggaataatcctaaaa

SEQ ID NO: 14 - DNA, 123 bp, Rattus sp.
caatacatctgcatcttgaccttttgttttcctcaaatatttcatctctgggccctgtctgttacagggttaccaggagtcaaattttatctaccataaatattcacatgaaaatagtaactt

SEQ ID NO: 15 - DNA, 117 bp, Rattus sp.
caatgcatttacagtctttattgtaaagacgttgcagttgtgacaggggacactgaaaatacacaatcacggattgttctttcagaagagagaggaatagatcccatatatacaaaa

SEQ ID NO: 16 - DNA, 104 bp, Rattus sp.
gataagcttcctgcaatggtacacccagacccagcacaagatgagaagacccagcttgttattaaaagacatcctcaagtgcatgttggttgtgtttggagtgt

SEQ ID NO: 17 - DNA, 116 bp, Rattus sp.
cctgcaaatgcaaacaatgcaaatgcacctcctgcaagaaaagctgctgttcctgctgccccgtggtgtgcgaagtgctcccagggctgcatctgcaaagaggcttcggacaagtg

SEQ ID NO: 18 - DNA, 68 bp, Rattus sp.
cctgcaaatgcaaacaatgcaaatgcacctcctgcaagaaaagctgctgttcctgctgccccgtggtg

SEQ ID NO: 19 - DNA, 176 bp, Rattus sp.
ggccttacgaatgttaacagtttcaactatacatgagaacgacacaacaacatttagtttgcttatgaacttcataaaagaacatgtttaatcttcattcttgaaatccatttcattgcattgtcaaatgataatttcagtgtagcttcacaacaacaacacatgctccagctctt

SEQ ID NO: 20 - DNA, 108 bp, Rattus sp.
ttagcagcagcagcagaataccacagcaggaaaatgaagagctggcaacagcaggctctaggacatgtatataatgcactgctattgtacagtacagagagaaagact

SEQ ID NO: 21 - DNA, 81 bp, Rattus sp.
  misc_feature (45)..(45): n is a, c, g, or t
ttagcagcacgaggagaaaacgggaggatgtgtctgaaaaaagcngtcctgcacagaagtattgtatttttatgaattttt

SEQ ID NO: 22 - DNA, 76 bp, Rattus sp.
cgtacgtatacacactcacacatacaaatttattcgaattcagcaaatagacccatcttagctaaaagaagaattc

SEQ ID NO: 23 - DNA, 159 bp, Rattus sp.
ccatgcatttgctaatttcattaccattaaagtttgttgcaacacaagtttccagaaattctaattttgtcttttaaacatttaccatcatcagtgaacacttatcagtgaaaggttcagaatgttcatttttcaggaacacagctaaatatccaagcc

SEQ ID NO: 24 - DNA, 202 bp, Rattus sp.
tccatgggaaacgaagcggaccagagtgcctatcaccacatctgggctatcagtagactgtccacaccttgtcttgctcttagcccctcggactccatccgtgttgccccctggtgtccacggaagactgtacagaaccattgtttatttatagccactctcccattggatttttaagaaaacaggataaacgatgatgttt

SEQ ID NO: 25 - DNA, 104 bp, Rattus sp.
acgttatataacaaatcaagtccatagtgagaacattattatggagaaatatgcaatggcttctgcataggacattcatatctatttgaagaagcttgtgatcc

SEQ ID NO: 26 - DNA, 100 bp, Rattus sp.
aaaaacaacaggagcaacaatgaaaaactaaaagtaaagacggtgacggatcagggcactaaaaaacttacagttgttgtataagctgcttctgggtcgt

SEQ ID NO: 27 - DNA, 99 bp, Rattus sp.
aagcttggtgaactggtagaagtgcgtcctgggagtgaaatgaggtgtcactggcaggtgagtgaaaacagcctgagtcttcccttggctagtaagccc

SEQ ID NO: 28 - DNA, 175 bp, Rattus sp.
atcattaaaacgccccagactttatttagcattagttagcaaagctggcttaacaaagtgaaagctgaaagttcacctagttaggaaaaggaacttacagatgagaaatgaaacactaggaaactcagaagcgacaaatccaagtggtaggagaaagcaaatgccctatacgtac

SEQ ID NO: 29 - DNA, 100 bp, Rattus sp.
ccctaatgtattgtgtagcaatacatggaaaggatcatggaagcatgacacaagatgtaatttaaattttcaaaagtcttatggttgtcatgaagagaga

SEQ ID NO: 30 - DNA, 101 bp, Rattus sp.
tacgtattgagttatgatttcaaatgctaccttgggaagttccaatggagatttgagttgaacagaaaaatacagtttcaggaacgtttcgttcttcttag

SEQ ID NO: 31 - DNA, 102 bp, Rattus sp.
  misc_feature (55)..(56): n is a, c, g, or t
ggtgaactgtaagtgggaaaaacagtgtaaaaatcagagatgatgtttaaaaaannttaaggctcttttgggacatgtgtgatatgctaggaaattaatttt

SEQ ID NO: 32 - DNA, 100 bp, Rattus sp.
ggtgaacagtaaaacaaatcaatagaacagaatggagtaatgaaccctcataaatacagtccacaaatcatttacaaagagcaaaggtgatctttccagc

SEQ ID NO: 33 - DNA, 100 bp, Rattus sp.
cttgcccacgtctcatacaaatgttctttgtcactttgtgagcagttgtgtgtatcctagtcttactcctacagcgtcaacatttgggaagccgagtcag

SEQ ID NO: 34 - DNA, 100 bp, Rattus sp.
aagcttgctgctagcaaaacaccagtagacacaaaataaaaataaagtataaaaaagaagacagggggctagagatatggctcagcgattgagagcactg

SEQ ID NO: 35 - DNA, 126 bp, Rattus sp.
  misc_feature (58)..(59): n is a, c, g, or t
atgcaaaggaaaagacgtgagggaaacggttgagcataatcaaaaaacacaatcctgnnccttaggttcctgtactgtacagcctcacatccagcccataggattcagtgaggagttaggtcttga

SEQ ID NO: 36 - DNA, 67 bp, Rattus sp.
ttggtgaacccagacggagataactcagaatgcttgtaaagtcagtccccacctgccccaaattttt

SEQ ID NO: 37 - DNA, 121 bp, Rattus sp.
ctgtcacagttatgatcaattccccgggaaagaacagcgcttttctaatttacaacagtcctccaaccgtctttacttaatcacgttttgaaatttccgtgtaaccgtttattaagcttct

SEQ ID NO: 38 - DNA, 86 bp, Rattus sp.
  misc_feature (72)..(72): n is a, c, g, or t
tttttttagttccaagtgtctggttatttcgcaatatcttttcggcgccgggcttgattagttgtgatatgngagtgaccgataaa

SEQ ID NO: 39 - DNA, 115 bp, Rattus sp.
tcgtcgtcgtcgtcaaggacatccaggtacccgagaaaccacttgtcgtgtgtggtaacaccgtcggcggaagtctcacgggttcggttcacaaaccaccctctttgttctcagc

SEQ ID NO: 40 - DNA, 79 bp, Rattus sp.
  misc_feature (51)..(52): n is a, c, g, or t
gtatctgcccaactaagtactttaacaaaaatccatcgagcaaaccaaagnncnaagaagtcgaatttaggaaaagcaa

SEQ ID NO: 41 - DNA, 147 bp, Rattus sp.
ccctttacaacgagatccatttaatctttaaaaaaaaagattatgatactttttccgttctcaaaaagagccagtcgtatgtaaaatataaatgaaacaggaaaatgacgtggggtctcttggaaaatactaattcccactaaaccg

SEQ ID NO: 42 - DNA, 91 bp, Rattus sp.
  misc_feature (79)..(80): n is a, c, g, or t
aacctgtgtcttaggcacactacgacatgggtagggcctaaatttgattacctctcatttatttattttcatctaaacnngagaacttttt

SEQ ID NO: 43 - DNA, 106 bp, Rattus sp.
caattttgcggggtctgaaataaatcgtaatcaatcgtttcgaccgaattgtttcactttcgactttcaagtgatcaatccttttccttgaatgtctactctttac

SEQ ID NO: 44 - DNA, 134 bp, Rattus sp.
  misc_feature (29)..(29): n is a, c, g, or t
cttattttagtacaagtaaataaaaaaangacgtagaatcttccgtattttaatttatacaacgttacattatttaagtaggtatgttcacgttcagaggtcgatatcacgtaaaataccgtctaaataagtaa

SEQ ID NO: 45 - DNA, 85 bp, Rattus sp.
gccatggggaggatgagggacgactgaatgcctgactccaggtttgtggagatgtagtaattgctttccggtgaagctttgtata

SEQ ID NO: 46 - DNA, 95 bp, Rattus sp.
cattcaggggtatggtatgtttaggatgttttcaagttccccaaaccagatgttaagagaaataaaaatgtaaagtataaacaatgcttaaaaaa

SEQ ID NO: 47 - DNA, 126 bp, Rattus sp.
caggagagaatcgacattgttggagtcaagtgtgtcactgtataaggttggaaccctccctctcccacctaaggaagctcaggcagaggagaagggaaacagacaaatttcatgaaatcagccagg

SEQ ID NO: 48 - DNA, 122 bp, Rattus sp.
gtatgtggatgtcacctattctaatactacttgctggaaagatcacctttcctctttgtaaatgatttgtggactgtatttatgagggttcattactccattctgttctattgatttgcttt